(19) United States
(12) Patent Application Publication
(10) Pub. No.: US 2024/0011392 A1
(43) Pub. Date: Jan. 11, 2024

(54) FAILURE CRITERION SELECTION BASED ON A STATISTICAL ANALYSIS OF FINITE ELEMENT MODEL RESULTS AND ROCK IMAGING DATA FOR WELLBORE MODELLING

(71) Applicant: Saudi Arabian Oil Company, Dhahran (SA)

(72) Inventor: Hussain AlBahrani, Al Qatif (SA)

(21) Appl. No.: 17/857,826

(22) Filed: Jul. 5, 2022

(51) Int. Cl. E21B 49/00; G06F 30/23

(52) U.S. Cl. CPC E21B 49/005; G06F 30/23; E21B 2200/20; G06F 2119/02

(57) ABSTRACT

Systems and methods for determining a failure criterion for a rock and using the failure criterion to determine a structural integrity of a wellbore include one or more of the following features. Some systems extract a core sample of the rock from the wellbore and acquiring images of the core sample. Some systems generate a finite element model of the core sample that includes a failure criterion, solve the model to generate nodal data, and use the nodal data to determine failure regions of the core sample. Some systems compare the images of the core sample to the failure regions of the core sample to determine confusion matrix parameters associated with nodes of the model. Some systems select the failure criterion based on an overall score of the confusion matrix parameters, and use the selected failure criterion to predict a structural integrity of the wellbore.

### TECHNICAL FIELD

The present disclosure relates to systems and methods for failure criterion selection using a statistical analysis based on finite element model results and rock imaging data for wellbore modelling (for example, wellbore integrity modelling, formation modelling, etc.).

### BACKGROUND

Quantitative description of wellbore failure is conventionally provided using mechanical multi-arm caliper logs, which provide radial measurements at four circumferential angles (0°, 90°, 180°, 270°) in their most common designs. On the other hand, drilling operations and supporting lab-based activities produce a substantial amount of data in the form of images. In the field, imaging tools are routinely run into the wellbore to extract information about the subterranean rock formations being drilled in the form of image logs. These imaging tools have a lot of variations in terms of imaging techniques and the format of produced images. The most common image logs are either resistivity based or ultrasonic-based. In laboratory environments, when rock core samples are tested for the purpose of measuring their mechanical properties and strength, different imaging techniques can be utilized to monitor the progression of failure in the rock matrix.

### SUMMARY

The systems and methods of this disclosure compare the results of a three-dimensional finite element model to reference data to determine a failure criterion that represents rock failure. In some cases, the reference data are images of a core sample that has been extracted from a wellbore. In some cases, the reference data is logging data that has been acquired from the wellbore. Data is output from the finite element model at each node of the finite element model and is compared with the reference data to determine whether the failure predicted by the finite element model is accurate. The accuracy of the finite element model is determined by generating a confusion matrix indicating whether each node of the finite element model accurately predicts failure (e.g., true positive), whether each node accurately predicts non-failure (e.g., true negative), whether each node incorrectly predicts failure (e.g., false positive), and whether each node incorrectly predicts non-failure (e.g., false negative). These results are used as part of a statistical analysis that gives equal weight to the entire finite element model to reduce or remove bias of results towards the wellbore outer diameter (for example, where more nodes may be present). The statistical analysis determines the failure criterion that represents the rock failure in the wellbore while reducing or removing this bias.

After the failure criterion is determined, the failure criterion is used in a three-dimensional finite element model to predict rock failure within the wellbore under loading conditions representative of a downhole environment of the wellbore. In some examples, the finite element model is solved to predict whether the wellbore is over-pressurized or under-pressurized when a particular mud weight is used. In some examples, the finite element model is solved to determine a range of mud weights or a particular mud weight that should be pumped in the wellbore to stabilize the well. In some examples, the systems and methods of this disclosure pump a mud having the determined mud weight into the well to stabilize the well to reduce the likelihood that the wellbore becomes over-pressurized or under-pressurized.

The present disclosure describes computer-implemented methods, computer-readable media, and systems for converting images into three dimensional structures for numerical modeling and simulation applications. Additional details related to converting images into three dimensional structures for numerical modeling and simulation applications are described in U.S. patent application Ser. No. 17/644,135 filed Dec. 14, 2021 and is incorporated herein by reference in its entirety.

One example computer-implemented method includes receiving multiple CT-scan images of a core sample of a rock, where the core sample of the rock includes a borehole, and the multiple CT-scan images are cross-section CT-scan images of the core sample of the rock at multiple depths of the wellbore (or borehole). A triangulation process is performed on pixels of each of the multiple CT-scan images for each of the multiple CT-scan images and with respect to each of multiple circumferential position angles. Multiple radii of the borehole corresponding to the multiple circumferential position angles are determined for each of the multiple CT-scan images. Multiple nodal coordinates of 3D numerical model mesh of the borehole are generated based on the multiple radii of the borehole of each of the multiple CT-scan images and using a meshing function. An advisory on drilling window limits of mud weight is provided for avoiding rock failure of a wellbore, based on the multiple nodal coordinates of the 3D numerical model mesh of the borehole.

With the recent advancements in image analysis, there lies a potential to utilize the different forms of available images for extracting valuable information for drilling engineering applications. Different image logs from the field and different images from lab experiments can help inform drilling geomechanics models of the patterns and limits of a borehole rock failure. These images can be used to construct three-dimensional (3D) structures before and after rock failure takes place. These structures can then be converted to 3D meshes, which in turn can be employed within a numerical model, such as a finite element model, to estimate the stress distribution around the structure. The end result of this process is a more accurate depictions of the drilled structures and a more accurate prediction of the rock failure limits. On the other hand, conventionally quantitative description of wellbore failure may not provide a comprehensive description of the wellbore shape as well as images can, as mechanical multi-arm caliper logs usually only provide radial measurements at four circumferential angles (0°, 90°, 180°, 270°) in their most common designs.

This disclosure describes technologies for implementing a conversion from borehole images into three dimensional structures for numerical modeling and simulation applications. In some implementations, image analysis techniques can provide the ability to quantitatively determine the extent of wellbore wall rock failure from images. The quantitative description of failure from images can be used to provide a node-based classification of rock failure, which can be used to correlate the classification to each separate node in the numerical model mesh. By providing this link to the numerical model, a more accurate mapping of actual failures is now possible. The end result is an integrated finite element model and machine learning (ML) model, which is trained based on offset wells data or previous lab experiments to predict the limits for rock failure in new wells or experiments.

In some implementations, image analysis techniques can enable numerical models to reflect the effect of different drilling events as they occur through images produced from logging while drilling (LWD) tools. This can ensure that events that led to changes to the wellbore geometry and shape can be reflected in the model mesh. This while-drilling consideration of changes to the wellbore shape and structure can enable the rock strength limits and probability of failure be updated as drilling progresses.

Some systems and methods for determining a failure criterion and using the failure criterion to determine a structural integrity of a wellbore include one or more of the following features. Some systems and methods extract a core sample of a rock from the wellbore. Some systems and methods acquire one or more images of the core sample after the core sample has been subject to specified loading conditions using a tri-axial testing machine. Some systems and methods generate a finite element model of the core sample. The finite element model includes a constitutive model and a failure criterion. Some systems and methods solve the finite element model of the core sample to generate nodal data indicating whether each node of one or more nodes of the finite element model is predicted to fail or not while being subject to the specified loading conditions, the constitutive model, and the failure criterion. Some systems and methods use the nodal data to determine one or more predicted failure regions of the core sample and one or more predicted non-failure regions of the core sample based on whether each node is predicted to fail or not.

Some systems and methods compare the one or more images of the core sample to the one or more predicted failure regions of the core sample and to the one or more predicted non-failure regions of the core sample to determine one or more confusion matrix parameters associated with the one or more nodes of the finite element model. Some systems and methods determine an overall score of the failure criterion based on the one or more confusion matrix parameters associated with the one or more nodes of the finite element model. Some systems and methods select the failure criterion based on the overall score and use the selected failure criterion to predict the structural integrity of the wellbore. Some systems and methods select a mud weight based on the predicted structural integrity of the wellbore and pump the mud having the selected mud weight into the wellbore to provide structural stability to the wellbore.

Some systems and methods determine the one or more confusion matrix parameters associated with the one or more nodes of the finite element model by determining whether each confusion matrix parameters is true negative, false negative, false positive, or true positive.

Some systems and methods determine the overall score of the failure criterion based on the one or more confusion matrix parameters associated with the one or more nodes of the finite element model by: (i) determining an overall accuracy of the failure criterion based on the one or more confusion matrix parameters, (ii) determining an overall recall of the failure criterion based on the one or more confusion matrix parameters, and (iii) determining the overall score of the failure criterion based on the precision and the recall. In some cases, the overall score is independent of a skewness of the one or more confusion matrix parameters toward a particular true negative, false negative, false positive, or true positive result.

Some systems and methods compare the one or more images of the core sample to the one or more predicted failure regions of the core sample and the one or more predicted non-failure regions of the core sample to determine the one or more confusion matrix parameters associated with the one or more nodes of the finite element model by: (i) perturbing the one or more nodes of the finite element model based on the acquired one or more images, and (ii) comparing the perturbed finite element model to the one or more predicted failure regions of the core sample and the one or more predicted non-failure regions of the core sample to determine the one or more confusion matrix parameters associated with the one or more nodes of the finite element model.

Some systems and methods compare the one or more images of the core sample to the one or more predicted failure regions of the core sample and the one or more predicted non-failure regions of the core sample to determine the one or more confusion matrix parameters associated with the one or more nodes of the finite element model by superimposing the one or more predicted failure regions of the core sample on the one or more images of the core sample.

Some systems and methods compare the one or more images of the core sample to the one or more predicted failure regions of the core sample and the one or more predicted non-failure regions of the core sample to determine the one or more confusion matrix parameters associated with the one or more nodes of the finite element model by: (i) determining a first set of nodes of the finite element model that are associated with the one or more predicted failure regions of the core sample and associated with one or more failure regions of the one or more images of the core sample, (ii) determining the confusion matrix parameter for the first set of nodes as true positive, (iii) determining a second set of nodes of the finite element model that are associated with the one or more predicted non-failure regions of the core sample and associated with one or more non-failure regions of the one or more images of the core sample, and (iv) determining the confusion matrix parameter for the second set of nodes as true negative.

Some systems and methods compare the one or more images of the core sample to the one or more predicted failure regions of the core sample and the one or more predicted non-failure regions of the core sample to determine the one or more confusion matrix parameters associated with the one or more nodes of the finite element model by: (i) determining a third set of nodes of the finite element model that are associated with the one or more predicted failure regions of the core sample and associated with the one or more non-failure regions of the one or more images of the core sample, (ii) determining the confusion matrix parameter for the third set of nodes as false positive, (iii) determining a fourth set of nodes of the finite element model that are associated with the one or more predicted non-failure regions of the core sample and associated with the one or more failure regions of the one or more images of the core sample, and (iv) determining the confusion matrix parameter for the fourth set of nodes as false negative.

Some systems and methods triangulate pixels of each of the one or more images. In some examples, each of the one or more images is a cross-section image of the core sample where the cross-section is perpendicular to a longitudinal axis of the core sample. Some systems and methods determine a plurality of radii of the core sample based on the triangulated pixels. In some examples, the plurality of radii of the core sample is a function of circumferential position around the longitudinal axis of the core sample. Some systems and methods compare the one or more images of the core sample to the one or more predicted failure regions of the core sample and to the one or more predicted non-failure regions of the core sample by determining the first set of nodes of the finite element model, the second set of nodes of the finite element model, the third set of nodes of the finite element model, and the fourth set of nodes of the finite element model based on the plurality of radii of the core sample.

Some systems and methods adjust a contrast of each of the one or more images such that that a wellbore void in each of the one or more images is a first color, and a rock matrix surrounding the wellbore void in each of the one or more images is a second color that is different than the first color. In some cases, after adjusting the contrast of each of the one or more images, the systems and methods calibrate a size of each of the one or more images based on an actual pixel size measurement from a reference image. Some systems and methods compare the one or more images of the core sample to the one or more predicted failure regions of the core sample and to the one or more predicted non-failure regions of the core sample by comparing the one or more images of the core sample to the one or more predicted failure regions of the core sample and to the one or more predicted non-failure regions of the core sample based on the calibrated size of each of the one or more images.

Some systems and methods triangulate the pixels of each of the one or more images separately along horizontal and vertical directions and then combine the horizontal and vertical directions to define the triangulated the pixels.

Some systems and methods repeat the steps of: (i) generating the finite element model of the core sample, (ii) solving the finite element model to generate the nodal data, (iii) using the nodal data to determine the one or more predicted failure regions of the core sample and the one or more predicted non-failure regions of the core sample, (iv) comparing the one or more images of the core sample to the one or more predicted failure regions of the core sample to determine one or more confusion matrix parameters associated with the one or more nodes of the finite element model, and (v) determining the overall score of the failure criterion based on the one or more confusion matrix parameters associated with the one or more nodes of the finite element model. In some cases, steps (i)-(v) are repeated for a plurality of different failure criterions to determine an overall score associated with each of the plurality of different failure criterions. In some cases, the plurality of different failure criterions including at least two of a Mohr-Coulomb failure criterion, a Mogi failure criterion, a Mogi-Coulomb criterion, and a Drucker-Prager failure criterion. Some systems and methods select the failure criterion based on the overall score by selecting the failure criterion based on a ranking of the overall scores for the plurality of different failure criterions.

Some systems and methods use the selected failure criterion to predict the structural integrity of the wellbore by: (i) generating a three-dimensional finite element model of the wellbore, the three-dimensional finite element model of the wellbore including the constitutive model and the selected failure criterion, (ii) solving the three-dimensional finite element model of the wellbore to generate nodal data indicating whether each node of one or more nodes of the finite element is predicted to fail or not while being subject to the selected failure criterion, (iii) using the nodal data of the three-dimensional finite element model of the wellbore to determine one or more predicted failure regions of the wellbore and one or more predicted non-failure regions of the wellbore based on whether each node is predicted to fail or not, and (iv) predicting the structural integrity of the wellbore based on the one or more predicted failure regions of the wellbore.

Some systems and methods acquire a stress-strain curve of the core sample while the core sample is subjected to one or more loading conditions using the tri-axial testing machine. Some systems and methods determine an unconfined compression strength of the core sample based on the acquired stress-strain curve. In some cases, the constitutive model represents the determined unconfined compression strength.

Some systems and methods acquire logging data of the rock. In some cases, the logging data includes a vertical stress of the rock, a horizontal stress of the rock, image log data of the rock, and a depth within the wellbore associated with the acquisition of the logging data. Some systems and methods predict an unconfined compression strength of the rock based on the acquired logging data. In some cases, the constitutive model further represents the determined unconfined compression strength.

Some systems and methods acquire the one or more images of the core sample after the core sample has been subject to specified loading conditions using the tri-axial testing machine by performing a CT-scan on the core sample to generate the one or more images of the core sample.

Some systems and methods for determining a failure criterion and using the failure criterion to determine a structural integrity of a wellbore include one or more of the following features. Some systems and methods acquire logging data of a rock surrounding the wellbore using a logging tool within the wellbore. In some cases, the logging data includes a vertical stress of the rock, a horizontal stress of the rock, image log data of the rock, and a depth within a wellbore associated with the acquisition of the logging data of the rock. Some systems and methods predict an unconfined compression strength of the rock based on the acquired logging data. Some systems and methods generate a finite element model of the wellbore based on the acquired logging data. In some cases, the finite element model of the wellbore includes a constitutive model and a failure criterion that accounts for the unconfined compression strength of the rock. Some systems and methods solve the finite element model of the wellbore to generate nodal data indicating whether each node of the one or more nodes of the finite element model is predicted to fail or not while being subject to specified loading conditions. In some cases, the constitutive model and the failure criterion that accounts for the unconfined compression strength of the rock. Some systems and methods use the nodal data to determine one or more predicted failure regions of the wellbore and one or more non-failure regions of the wellbore based on whether each node is predicted to fail or not.

Some systems and methods compare one or more images of the rock from the image log data to the one or more predicted failure regions of the rock of the wellbore and to the one or more predicted non-failure regions of the rock of the wellbore to determine one or more confusion matrix parameters associated with the one or more nodes of the finite element model. Some systems and methods determine an overall score of the failure criterion based on the one or more confusion matrix parameters associated with the one or more nodes of the finite element model. Some systems and methods select the failure criterion based on the overall score and use the selected failure criterion to predict the structural integrity of the wellbore. Some systems and methods select a mud weight based on the predicted structural integrity of the wellbore and pump the mud having the selected mud weight into the wellbore to provide structural stability to the wellbore.

Some systems and methods compare the one or more images of the rock from the image log data to the one or more predicted failure regions of the rock of the wellbore and to the one or more predicted non-failure regions of the rock of the wellbore to determine the one or more confusion matrix parameters associated with one or more nodes of the finite element model by: (i) determining a first set of nodes of the finite element model that are associated with the one or more predicted failure regions of the rock of the wellbore and associated with one or more failure regions of the one or more images of the rock from the image log data, (ii) determining the confusion matrix parameter for the first set of nodes as true positive, (iii) determining a second set of nodes of the finite element model that are associated with one or more predicted non-failure regions of the rock of the wellbore and associated with one or more non-failure regions of the one or more images of the rock from the image log data, (iv) determining the confusion matrix parameter for the second set of nodes as true negative, (v) determining a third set of nodes of the finite element model that are associated with the one or more predicted failure regions of the rock of the wellbore and associated with the one or more non-failure regions of the one or more images from the image log data, (vi) determining the confusion matrix parameter for the third set of nodes as false positive, (vii) determining a fourth set of nodes of the finite element model that are associated with the one or more predicted non-failure regions of the rock of the wellbore and associated with the one or more failure regions of the one or more images from the image log data, and (viii) determining the confusion matrix parameter for the fourth set of nodes as false negative.

Some systems and methods acquire the logging data of the rock using the logging tool within the wellbore comprises using a micro-imager or micro-scanner tool to acquire resistivity based images of the rock of the wellbore. In some cases, the failure criterion includes at least one of a Mohr-Coulomb failure criterion, a Mogi failure criterion, a Mogi-Coulomb criterion, and a Drucker-Prager failure criterion.

The systems and methods described in this specification provide one or more of the following advantages.

The systems and methods of this disclosure determine a failure criterion in a manner that is independent of a skewness of the results of the finite element model. For example, by representing the results of the finite element model in a confusion matrix, the skewness of results towards the wellbore outer diameter (the sidewall) are reduced/eliminated. This improves the accuracy of the determined failure criterion which might otherwise be biased towards the wellbore outer diameter (for example, where the number of nodes (or mesh density) might be higher than a region further away from the wellbore outer diameter).

The systems and methods of this disclosure determine a failure criterion in an automatic manner without the need for an engineer to intervene in the failure criterion determination. For example, the systems and methods automatically loop over each node of the finite element model and determine confusion matrix parameters for each node. The systems and methods automatically determine an F1-score for each failure criterion based on the confusion matrix parameters of all the nodes in the finite element model. The failure criterion with the highest F1-score is automatically selected by the systems and methods without engineer intervention. This automatic process reduces engineer bias and results in an objective process rather than a subjective process where the results could vary from engineer to engineer.

While generally described as computer-implemented software embodied on tangible media that processes and transforms the respective data, some or all of the aspects may be computer-implemented methods or further included in respective systems or other devices for performing this described functionality. The details of these and other aspects and implementations of the present disclosure are set forth in the accompanying drawings and the description below. Other features, objects, and advantages of the disclosure will be apparent from the description and drawings, and from the claims.

### DESCRIPTION OF DRAWINGS

FIG. 1A is an example of an over-pressurized wellbore. FIG. 1B is an example of an under-pressurized wellbore.

FIG. 2 illustrates an example CT-scan of a bored core sample showing the development of failure zones around the borehole, in accordance with example implementations of this disclosure.

FIG. 3 illustrates example digital image correlation (DIC) images showing the progress of rock failure, in accordance with example implementations of this disclosure.

FIGS. 4A-4D illustrate an example process of triangulation used to calculate the borehole radii from images, in accordance with example implementations of this disclosure.

FIG. 5 illustrates example full radial measurements of a CT-scan of a failed borehole as a result of combining the horizontal and the vertical triangulation readings and the spline smoothing step, in accordance with example implementations of this disclosure.

FIGS. 6A-C illustrate two example CT-scans and the superimposed radii measurements, in accordance with example implementations of this disclosure.

FIG. 7 illustrates an example mapping of the location and magnitude of borehole failure points, in accordance with example implementations of this disclosure.

FIG. 8A and FIG. 8B illustrate an example flow chart of a CT-scan borehole interpretation algorithm, in accordance with example implementations of this disclosure.

FIG. 9 illustrates an example of an ultrasonic image log from a depth location of a wellbore showing stress-induced wellbore enlargements, in accordance with example implementations of this disclosure.

FIG. 10A and FIG. 10B illustrate an example flow chart of an algorithm for calibrating ultrasonic image log interpretations using multi-arm caliper measurements, in accordance with example implementations of this disclosure.

FIG. 11A and FIG. 11B illustrate an example flow chart of an algorithm for applying the calibrated ultrasonic image log model, in accordance with example implementations of this disclosure.

FIG. 12 illustrates an example plot of the RGB numbers from an ultrasonic image log showing a correlation between the location of enlargements in the image and the trends in the RGB numbers, in accordance with example implementations of this disclosure.

FIG. 13 illustrates example results of the ultrasonic image log interpretation showing radial measurements in Cartesian and polar coordinates, in accordance with example implementations of this disclosure.

FIG. 14 illustrates example mechanical multi-arm caliper log readings and corresponding resistivity-based image log, in accordance with example implementations of this disclosure.

FIGS. 15A-15C illustrate an example flow chart of an algorithm for producing interpretations from resistivity image logs, in accordance with example implementations of this disclosure.

FIG. 16 illustrates an example original RGB number of an resistivity image log and the adjusted RGB values to account for the image gaps and other image noise, in accordance with example implementations of this disclosure.

FIG. 17 illustrates example results of the image log interpretations that are calibrated with mechanical caliper log readings in both Cartesian and polar coordinates, in accordance with example implementations of this disclosure.

FIGS. 18A-18C illustrate an example process of converting the borehole shape interpretations from field and lab images into a mesh nodal coordinate, in accordance with example implementations of this disclosure.

FIG. 19 illustrates an example meshing function ability to reflect different borehole shapes based on CT-scan taken across different cross-sections of the core sample, in accordance with example implementations of this disclosure.

FIG. 20 illustrates an example definition of key-seating and the results of the meshing function, in accordance with example implementations of this disclosure.

FIG. 21 illustrates an example meshing of a static fracture, in accordance with example implementations of this disclosure.

FIG. 22 illustrates example plots of nodal coordinates of a mesh with finite boundary elements and a mesh with infinite boundary elements, in accordance with example implementations of this disclosure.

FIGS. 23A-23C illustrate an example mesh created for modelling core samples, in accordance with example implementations of this disclosure.

FIGS. 24A and 24B are illustrations of a three-dimensional finite element model for determining stresses in a formation surrounding a wellbore.

FIG. 25 is a flow chart of a driver computer code of the finite element model that includes the main subroutine and twelve subroutines.

FIG. 26 is a flow chart of a method to determine whether nodes of an element of the finite element model have failed based on one or more failure criterions.

FIG. 27 is an illustration of a comparison between one or more images of the core sample and failure/non-failure regions from a three-dimensional finite element model for determining confusion matrix parameters.

FIG. 28 is an illustration of a comparison among an initial contour of a borehole, a predicted contour of the borehole as predicted by solving a finite element model, and a true contour as determined from reference data.

FIGS. 29A and 29B are schematics of a core sample subject to tri-axial compression. FIG. 29C is an image of an example core sample.

FIG. 30 is a plot of confusion matrix evaluations for three different failure criteria based on core sample imaging reference data.

FIG. 31 is a plot of confusion matrix evaluations for three different failure criteria based on wellbore logging reference data.

FIG. 32 is a flowchart illustrating an example process for determining the failure criterion based on a maximum F1-score value.

FIG. 33 illustrates example recommendations of mud weights or downhole pressures required to prevent wellbore rock failure based on the output of the initial setup of a numerical model, in accordance with example implementations of this disclosure.

FIG. 34 illustrates an example of updating the wellbore shape and adjusting the structure of the numerical model mesh based on information from logging while drilling wellbore images or from shale shakers images, in accordance with example implementations of this disclosure.

FIG. 35 illustrates an example of updated recommendations of mud weights or downhole pressures required to prevent wellbore rock failure, in accordance with example implementations of this disclosure.

FIG. 36 illustrates example flow charts depicting the distinction between two processes to determine the pre-drilling mud weight and the while-drilling mud weight update, in accordance with example implementations of this disclosure.

FIG. 37 illustrates an example supervised training process that uses a numerical model output as training features and borehole image interpretations as training responses, in accordance with example implementations of this disclosure.

FIGS. 38A and 38B are a flowchart illustrating an example of a method for determining wellbore integrity based on a statistical analysis of finite element model results combined with rock imaging reference data.

FIGS. 39A and 39B are a flowchart illustrating an example of a method for determining wellbore integrity based on a statistical analysis of finite element model results combined with rock logging reference data.

FIG. 40 depicts an environment architecture of an example computer-implemented system that can execute implementations of the present disclosure.

### DETAILED DESCRIPTION

The systems and methods of this disclosure compare the results of a three-dimensional finite element model to reference data to determine a failure criterion that represents rock failure. In some cases, the reference data are images of a core sample that has been extracted from a wellbore. In some cases, the reference data is logging data that has been acquired from the wellbore. Data is output from the finite element model at each node of the finite element model and is compared with the reference data to determine whether the failure predicted by the finite element model is accurate. The accuracy of the finite element model is determined by generating a confusion matrix indicating whether each node of the finite element model accurately predicts failure (e.g., true positive), whether each node accurately predicts non-failure (e.g., true negative), whether each node incorrectly predicts failure (e.g., false positive), and whether each node incorrectly predicts non-failure (e.g., false negative). These results are used as part of a statistical analysis that gives equal weight to the entire finite element model to reduce or remove bias of results towards the wellbore outer diameter (for example, where more nodes may be present). The statistical analysis determines the failure criterion that represents the rock failure in the wellbore while reducing or removing this bias.

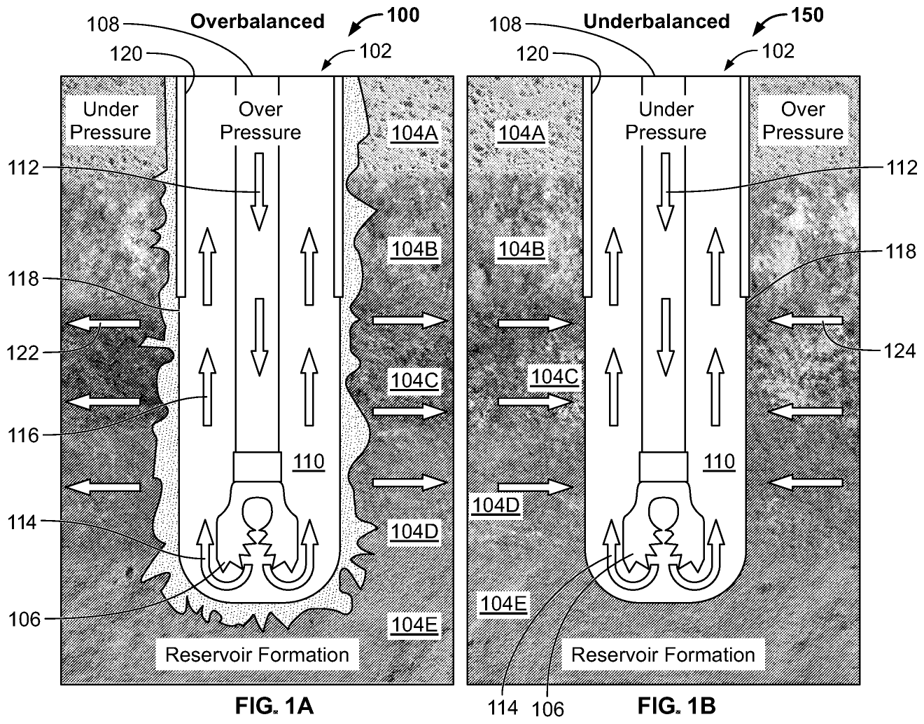

FIG. 1A depicts an example of an over-pressurized wellbore system 100 and FIG. 1B depicts an example of an under-pressurized wellbore system 150. The wellbore systems 100, 150 include a borehole or wellbore 102 into one or more layers of formation rock 104A-E (collectively referred to as “rock 104”). Each layer of the rock 104 is generally horizontal and has different geophysical properties (for example, density, shear modulus, compressive strength, etc.). At least one layer of rock 104 is permeable to hydrocarbons (for example, oil and gas) and is fluidly connected to an oil or gas reservoir so that oil and gas can be produced from the wellbore systems 100, 150 once the wellbore 102 is completed.

A drill bit 106 is rotationally coupled to a drill string 108 and is used to cut the rock 104 surrounding the wellbore 102. A drill (not shown) controls the rotational speed and vertical depth of the drill bit 106. Drilling fluid 110 (for example, drilling mud) is pumped into a central bore of the drill string 108 in the direction of arrow 112. The drilling fluid 110 exits the drill string 108 at the drill bit 106 in the direction of arrow 114 and recirculates back to the ground surface in the direction of arrow 116. A pump (for example, a hydraulic pump—not shown) pumps the drilling fluid 110 through the drill string 108 and recirculates the drilling fluid 110 returning to the ground surface. The returning drilling fluid 110 flows in an annular space between the drill string 108 and a wellbore outer diameter surface 118 (for example, the sidewall or wall of the wellbore 102). A casing 120 is located on a portion of the surface 118 to maintain the structural integrity of the wellbore 102 (for example, to assist in preventing a structural collapse of the wellbore due to an under-pressurization within the wellbore 102).

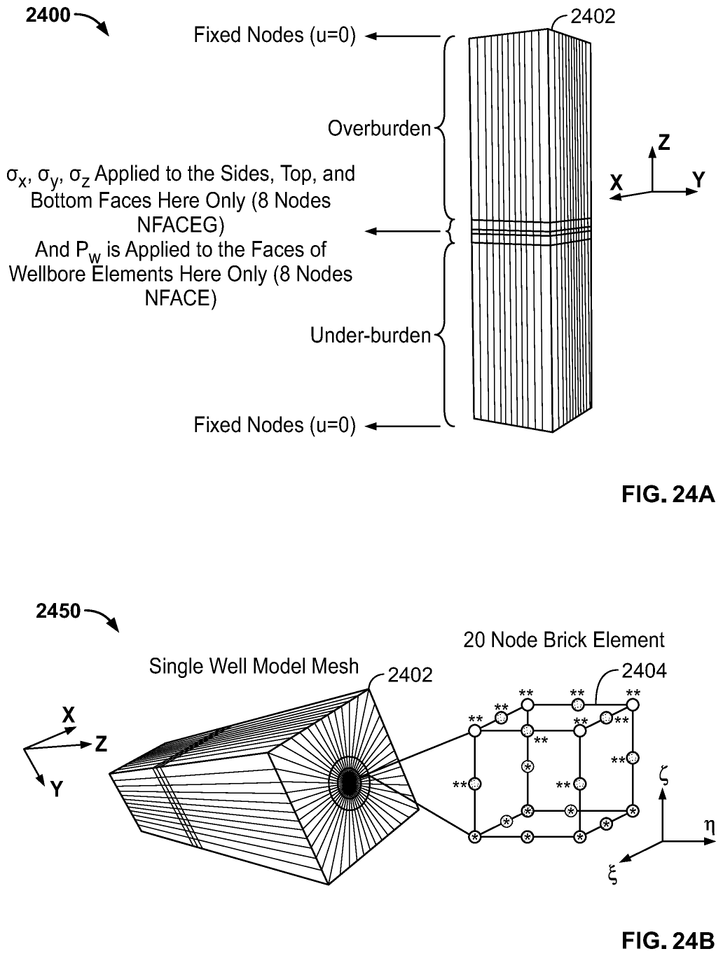

The drilling fluid 110 has geophysical properties (for example, density, viscosity, etc.) that are different from the geophysical properties of the rock 104. The drilling fluid 110 has a temperature below a dry cutting temperature of the drill bit 106 to assist in cooling the drill bit 106 while the drill bit 106 cuts through the rock 104. The drilling fluid 110 carries the rock cuttings in the flow of the drilling fluid 110 (for example, along the direction of arrow 116) to assist in removing the cuttings from the wellbore 102. The drilling fluid 110 has a density or weight (for example, a mud weight) larger than a first predetermined amount to prevent self-collapse of the wellbore 102 to assist in stabilizing the surface 118 of the wellbore 102 so that the wellbore 102 does not self-collapse. The drilling fluid 110 has a density lower than a second predetermined amount to prevent over-expansion of the wellbore 102 which could cause the rock 104 to fracture. In some examples, a finite element model is used to determine the necessary density or weight to prevent self-collapse and over-expansion of the wellbore 102 (for example, a finite element model determines the first and second predetermined amounts). Details about using finite element models to predict mud weights to prevent self-collapse and over-expansion of the wellbore 102 is described with reference to FIGS. 24A, 24B, 25, and 26.

In the example shown in FIG. 1A, the drilling mud 110 has a mud weight large enough to cause an over-pressurization of the wellbore 102. In some cases, this means that the mud weight is greater than the in-situ stresses of the rock 104. In the scenario shown in FIG. 1A, the drilling mud 110 fractures the rock 104 surrounding the wellbore 102 (for example, at the surface 118 and radially into the rock 104) even through a portion of the wellbore 102 includes a casing 120. The drilling fluid 110 seeps into the rock 104 in the direction of arrows 122. The scenario shown in FIG. 1A is bad because it can contaminate fresh water aquifers near the wellbore 102, can lead to a substantial loss of drilling fluid 110 which can be costly, and can displace oil and gas in a direction away from the wellbore 102 which is counterproductive to producing oil and gas from the wellbore 102.

In the example shown in FIG. 1B, the drilling mud 110 has a mud weight low enough to cause an under-pressurization of the wellbore 102. In some cases, this means that the mud weight is less than the in-situ stresses of the rock 104. In the scenario shown in FIG. 1B, the surface 118 of wellbore 102 is at risk of collapse due to a pressure exerted by the in-situ stresses of the rock 104 in the direction of arrow 124. This scenario is bad because it can lead to a complete loss of a wellbore 102 and permanent entrapment of the drill string 108 and drill bit 106 within the collapsed wellbore 102.

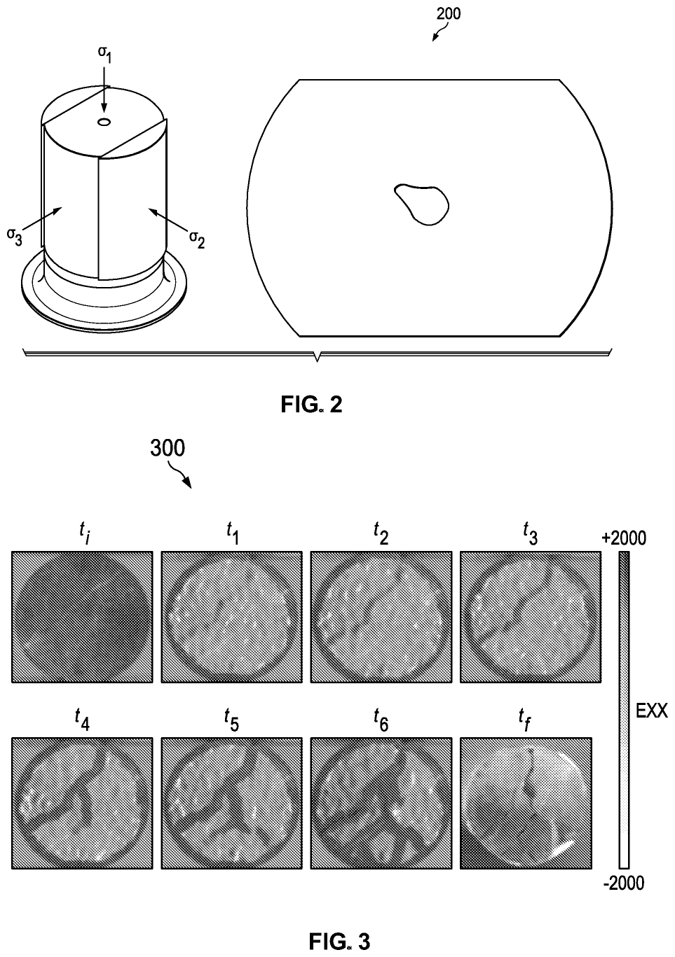

FIG. 2 illustrates an example CT-scan of a bored core sample 200 showing the development of failure zones around the borehole. In some implementations, the core sample 200 is extracted from the wellbore 102, a borehole is drilled through the core sample 200 to represent a wellbore being drilling in rock 104, and tested in a tri-axial testing machine to observe rock failure surrounding the borehole when subject to certain tri-axial testing conditions that mimic one or more conditions within the wellbore 102 (for example, temperature, in-situ stresses, pressures, etc.). The figure on the left of FIG. 2 illustrates an ideal perfectly cylindrical borehole and the figure on the right of FIG. 2 illustrates a borehole that has failed due to the one or more conditions represented by testing with the tri-axial testing machine. As a result, the borehole shown on the right of FIG. 2 is oblong shaped and is not perfectly circular (or cylindrical).

In some implementations, the core sample 200 is imaged while being tested in the tri-axial testing machine to generate closed-loop contours of the outer surface of the borehole as a function of depth within the borehole and time. In some implementations, lab-based images (for example, images acquired in a controlled laboratory setting), for example CT-scans or digital image correlation (DIC) images, can provide information regarding rock failure. In CT-scans, the location and the extent of rock failure around a borehole in the core sample (for example, the core sample 200) can be determined by visually examining the image and/or processing the images using a computer. For example, an imaging algorithm can process the CT-scans and/or DIC images to identify the location of failure and determine the magnitude of failure in terms of change to the borehole radius.

FIG. 3 illustrates example digital image correlation (DIC) images 300 showing the progress of rock failure. In some implementations, the imaging algorithm differentiates between the borehole and the rock matrix by first adjusting the contrast of the scans/images so that the borehole void is black colored, while the rock matrix is light gray colored. In FIG. 3, the dark regions represent void regions and the light regions represent rock regions. In some implementations, the data points in the image are pixels and the value for each point is the red-green-blue (RGB) color number.

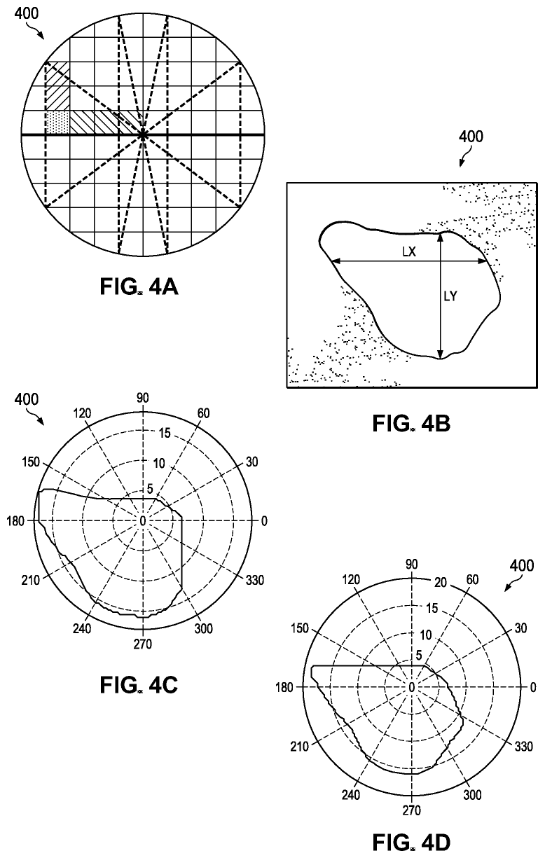

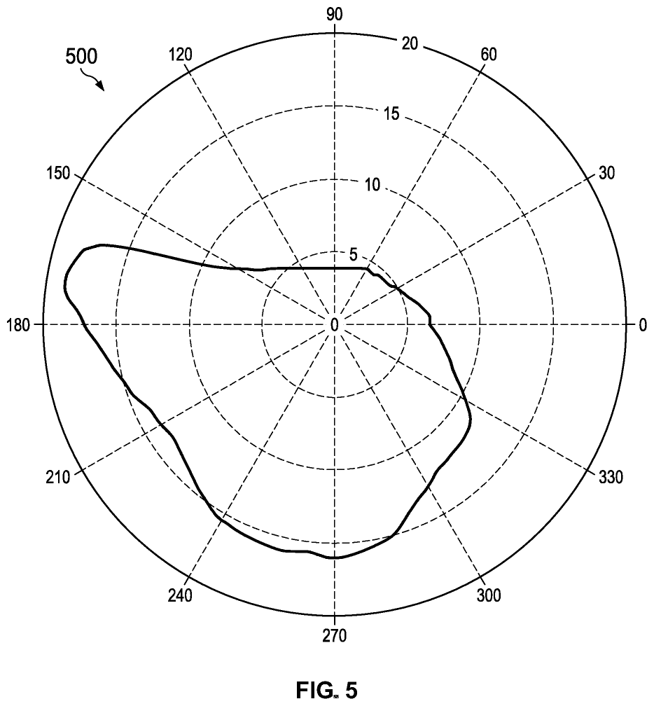

FIGS. 4A-4D illustrate an example process of triangulation used to calculate the borehole radii from images. The computer running the imaging algorithm processes the scans/images and triangulates the pixels to generate the plots 400. The information in these plots 400 are used to determine the closed-loop contour of the wellbore outer surface 118 as described with reference to FIG. 5. Through triangulation, a pixel that is centralized in the borehole can be selected, which can then be used as the first vertex of a triangle. Since the borehole shape in FIG. 4B is not circular, a center cannot be defined. Instead, this vertex is arbitrarily defined as the pixel that satisfies the intersection of the longest horizontal diameter (LX) with the longest vertical diameter (LY) of the non-uniformly shaped borehole as shown in FIG. 4B. From this vertex, horizontal and vertical straight lines are drawn by selecting the pixels from the defined vertex to the borehole wall. Then, a hypotenuse for a right triangle is formed at a specified angle. Next, the length of the hypotenuse is calculated using the Pythagorean theory. This calculated hypotenuse is one radial measurement, which is assigned to the circumferential position of the corresponding pixels. The total number of borehole pixels defining the adjacent and the opposite legs of the right triangle, as shown by the horizontal (HDim) and vertical (VDim) boxes in FIG. 4A, can be used in the Pythagorean theory calculation. This basic calculation is shown below:

Radial Measurement for One Circumferential Position=\sqrt{HDim²+VDim²}

In some implementations, this pixel-based measurement of length can be converted to an actual measurement of length by using the detected image resolution. This is done by using a reference CT-scan that depicts a uniform borehole with a known diameter. Next, the image pixels that define the borehole void, which are characterized by a black color RGB value, can be filtered out. Then, the total number of pixels in a line that intersects the borehole center can be determined. These values can be used in the followed formula to calculate the actual length of a pixel in mm:

$$Pixel Actual Length ( mm ) = \frac{2 \times Borehole Actual Radius ( mm )}{Total Number of Pixels in the LY Line}$$

In some implementations, the triangulation process is repeated both vertically and horizontally to provide full readings of the borehole radii as shown in FIG. 4C and FIG. 4D respectively. The number of the radii readings produced is dependent on the number of pixels available, i.e. image resolution, in the CT-scan image. Finally, the vertical and the horizontal radii readings are combined and a smoothing such as a spline smoothing can be applied to the curve to remove irregularities at the pixels corners as shown in FIG. 5.

FIG. 5 illustrates example full radial measurements of a CT-scan of a failed borehole as a result of combining the horizontal and the vertical triangulation readings and the spline smoothing step. The plot 500 shown in FIG. 5 represents the final result of the triangulation process described with reference to FIGS. 4A-4D and is a closed-loop contour representing the outer surface of the borehole of the core sample 200 at a particular depth within the borehole after the core sample 200 has been subject to tri-axial loading conditions that mimic one or more conditions of the downhole environment of the wellbore 102.

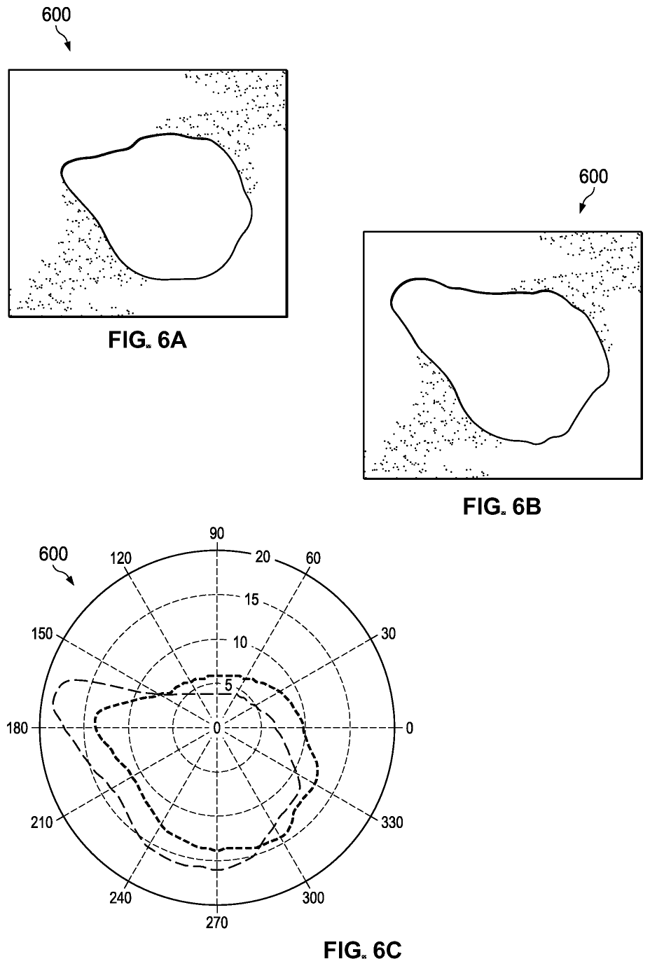

FIGS. 6A-6C illustrate two example CT-scans (plot 600 shown in FIGS. 6A and 6B) and the superimposed radii measurements (plot 600 shown in FIG. 6C). For example, 6A represents a cross-section of the borehole of the core sample 200 at a particular time during the tri-axial test and 6B represents the same cross-section at a later time during the tri-axial test. The imaging algorithm processes the sequence of scans/images to determine the extension of the failure zones from one CT-scan to the other. FIG. 6C is a composite image showing the closed-loop contour determined from the cross-section shown in FIG. 6A and the closed-loop contour determined from the cross-section shown in FIG. 6B.

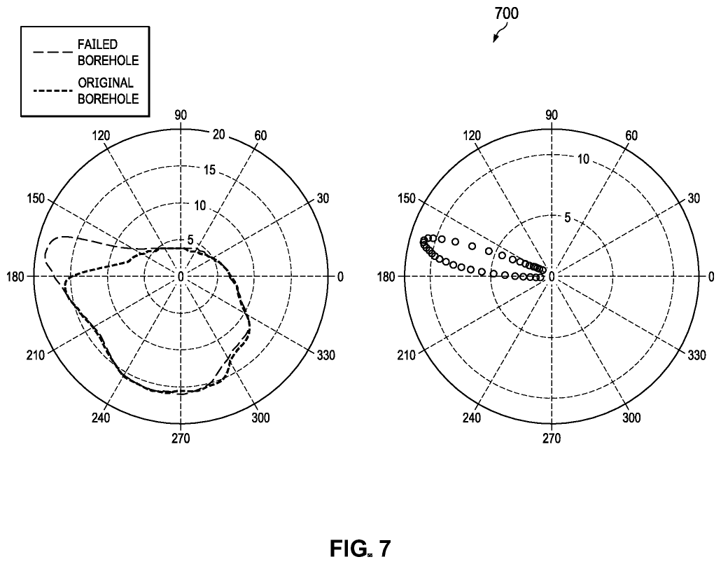

FIG. 7 illustrates an example plot 700 of a mapping of the location and magnitude of borehole failure points which generally represents the difference between two scans/images of a sequence of scans/images. For example, FIG. 7 shows the re-positioning of the two CT-scans from FIG. 6C to ensure the area of failure extension is accurately identified. FIG. 7 also shows a mapping of the failure extension zone, where the changes of radial measurements from one CT-scans to the other can be quantified at its specific circumferential location.

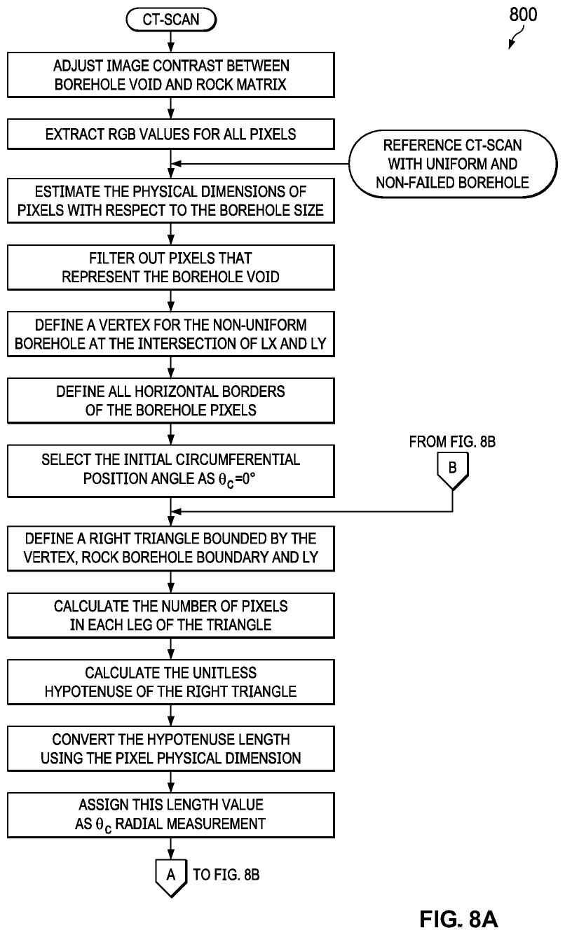

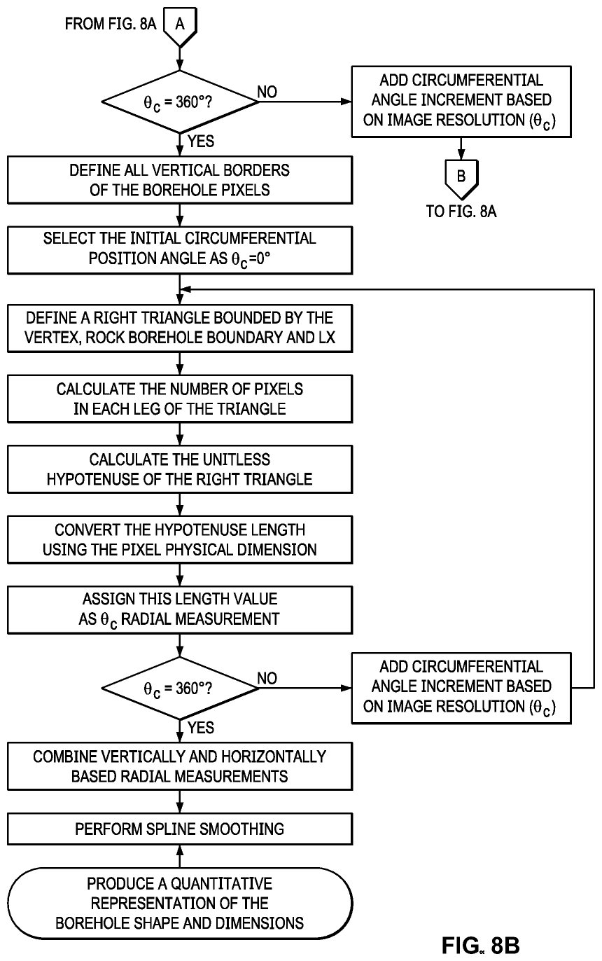

FIGS. 8A-8B illustrate an example flow chart 800 describing the imaging algorithm based on CT scans. Flow chart 800 generally represents the process described with reference to FIGS. 3, 4A-4D, 5, 6A-6C, and 7.

In some implementations, field-based images are used instead of, or in addition to, the lab-based images described above. For example, instead of, or in addition to extracting the core sample 200, imaging the core sample in a laboratory setting while subjecting the core sample 200 to downhole conditions in a tri-axial testing machine, and determine the closed-loop contours of the surface of the borehole from the lab-based images, the systems and methods described in this disclosure acquire imaging data from a wellbore (for example, wellbore 102) using logging data, process the logging data using a computer, and determine closed-loop contours of the surface of the wellbore from the field-based images. In some implementations, the imaging algorithm includes procedures for processing the CT-scans/DIC images as well as processing logging data to determine the closed-loop contours.

In some implementations, the imaging algorithm converts a specific type of imaging data (for example, ultrasonic based image logs), into caliper readings that cover a closed-loop around the wellbore circumference. The imaging algorithm logging data to produce radial measurements at all circumferential angles. The resolution of radial measurements is dependent on the image log resolution.

In some implementations, the ultrasonic image logs are preferred over resistivity-based image logs for at least two reasons. First, unlike the resistivity-based image logs, ultrasonic images have no gaps, and therefore, can provide a more comprehensive description of the wellbore state. Second, they are available in logging while drilling (LWD) tools in the form of density-based images, therefore they can be used in a real-time setting or a while-drilling setting to feed new information into numerical models.

In some implementations, a difference in the interpretation process between lab-based images (CT-scans/DIC images) and field-based image logs is that field-based image logs are generally not solely relied on to produce the interpretation. This is mainly due to the challenging environment in which these images are taken. This is also due to variations in image attributes from one commercial logging tool to the other. In some examples, field image logs require more support data and validation than lab-based images.

In some implementations, the support data is data from mechanical caliper logs, which provide radial measurements at four circumferential angles (for example, 0, 90, 180, and 270 degrees). For each image logging tool, the analysis of its images can be calibrated and validated against mechanical caliper measurements taken from the same interval where the images were produced. This ensures that the quantitative assessment of enlargements and tight spots from an image is validated using the mechanical caliper measurements of the same enlargements and tight spots. Once this process is performed, the calibrated algorithm can be used to interpret images from the same logging tool without running a mechanical caliper log every time an image log is produced.

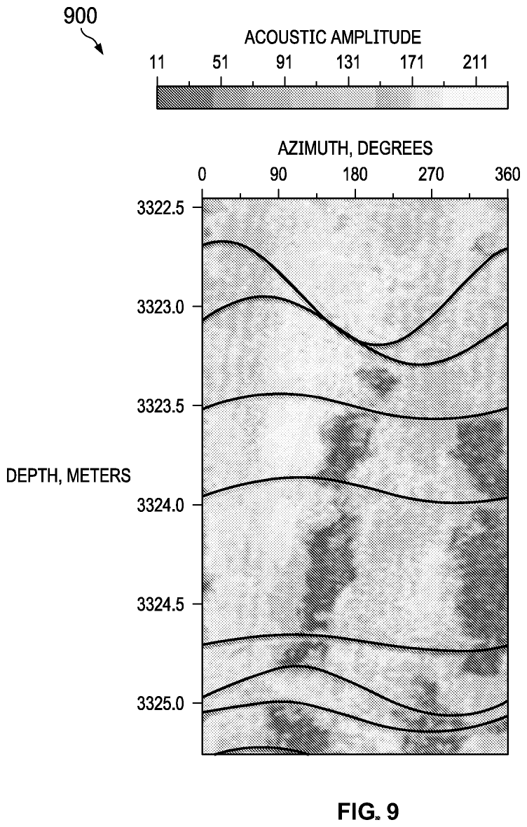

FIG. 9 illustrates an example plot 900 of an ultrasonic image log from a depth location of a wellbore showing stress-induced wellbore enlargements. In some implementations, this process is used for interpreting ultrasonic image logs by converting the RGB color number or values of each pixel to radial measurements. FIG. 9 illustrates an example of an ultrasonic image log. The area highlighted with a box in the middle section of FIG. 9 is the interval that is used to provide an example of an image log interpretation.

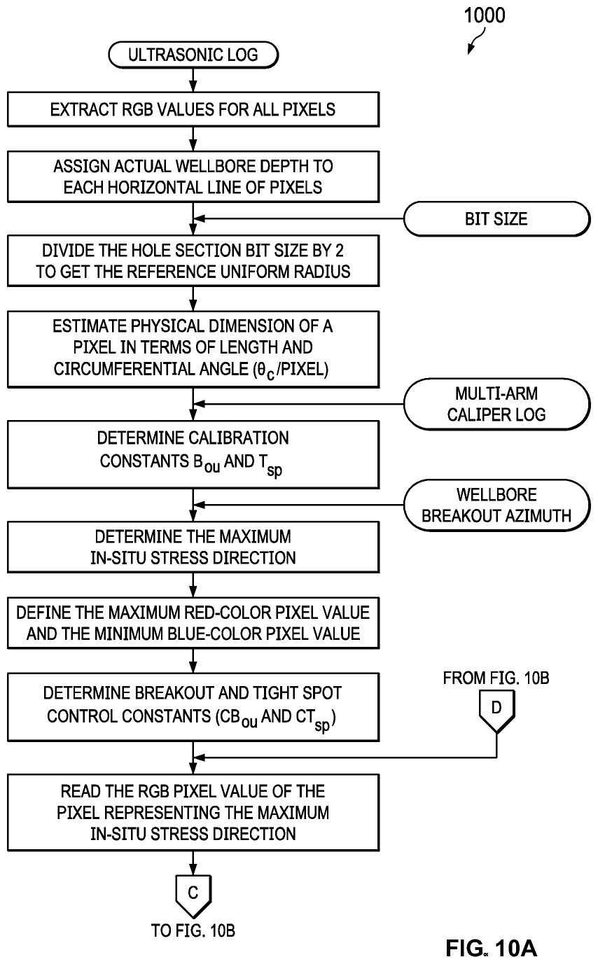

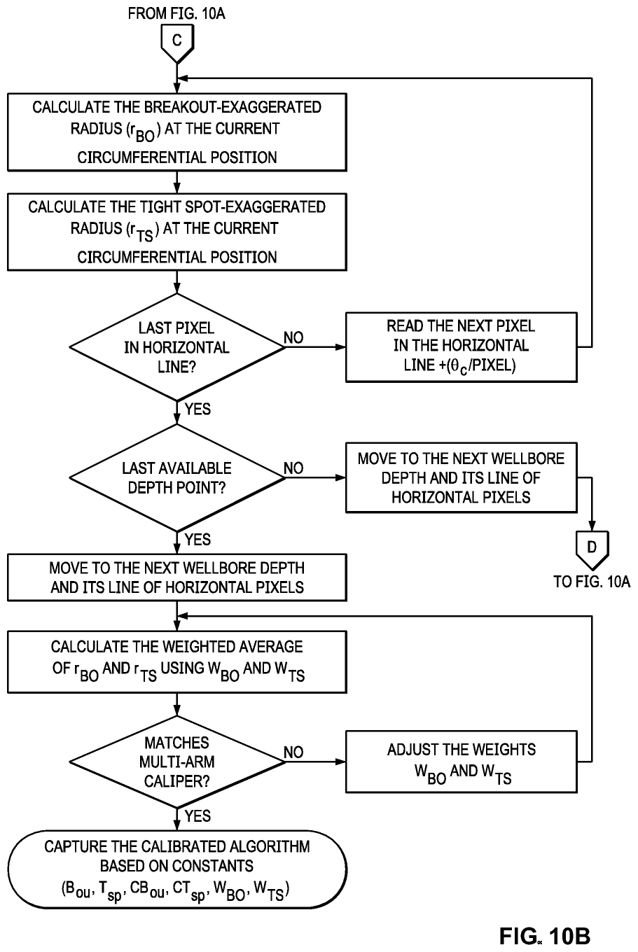

FIG. 10A and FIG. 10B illustrate an example flow chart 1000 of an imaging algorithm for calibrating the closed-loop contour determination based on ultrasonic image logs and mechanical caliper log data. In some examples, data other than multi-arm mechanical caliper log data is used. For example, bit size and/or a definition of the direction of the maximum in-situ stress are used. In some examples, the bit size is used to assign physical dimension to an individual pixel size. In some examples, the maximum in-situ stress direction is employed so that it can be correlated with the direction of wellbore enlargements or breakouts as observed in the image. The multi-arm mechanical caliper log measurements can be used to ascertain the extent of the radial depth of a darkly-colored breakout zone such as the ones shown as black in FIG. 9. They can also be used to ensure correct interpretation of tight spots, which are characterized by bright spots (shown as white in FIG. 9) on the log and a radius that is smaller than that of the bit size. This step determines Bₒᵤ and Tₛₚ, which are the breakout and the tight spot calibration constants respectively. The next step in the algorithm described in FIG. 10A is to extract the maximum red-color pixel value and the minimum blue-color pixel value. These two values can be used to determine the breakout and tight spot control constants (CBₒᵤ and CTₛₚ) as follows:

$${CB}_{ou} = \frac{Bit size}{Maximum red color pixel value} , and$$

$${CT}_{sp} = \frac{Bit size}{Minimum blue color pixel value} .$$

CBₒᵤ and CTₛₚ can be correlated to the identification of the most darkly colored region (shown as black in FIG. 9) in the image (which represents the deepest breakout) and the most brightly colored region (shown as white in FIG. 9) in the image (which represents the tightest spot in the hole). These constants, along with the calibration constants, can be used to calculate the breakout-exaggerated radii (r_{BO}) and the tight spot exaggerated-radii (r_{TS}) as follows:

r_{BO}=Bₒᵤ(Bit size−(Redᵢ×CBₒᵤ))+Bit size, and

r_{BO}=Bₒᵤ(Bit size−(Redᵢ×CBₒᵤ))+Bit size,

where Red and Blue r are the maximum and minimum values for all pixels in a single horizontal line which defines the borehole full circumference. The final step is the calculation of a weighted average between r_{BO} and r_{TS} as follows:

Radius=W_{BO}×r_{BO}+W_{TS}×r_{TS}

where W_{BO} and W_{TS} are the average weights, which are determined through a trial and error process that involves matching the weighted average with the actual multi-arm mechanical caliper log measurements as shown in FIG. 8A and FIG. 8B. This calibration algorithm needs to be applied to each unique image logging tool to ensure that the images produced from the tool are correctly interpreted. The same process may potentially need to be repeated for a calibrated tool when it is run in a new environment.

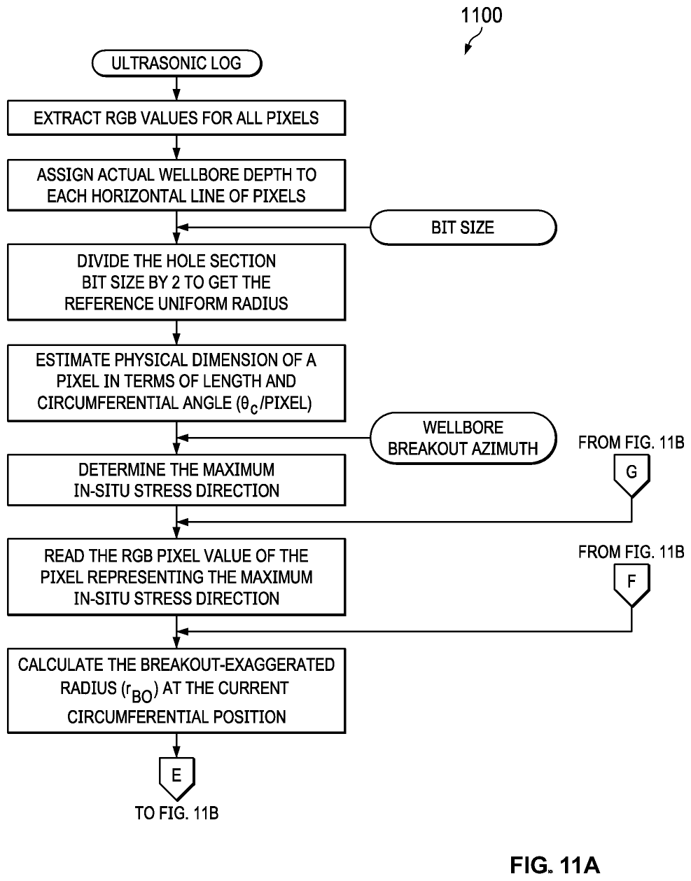

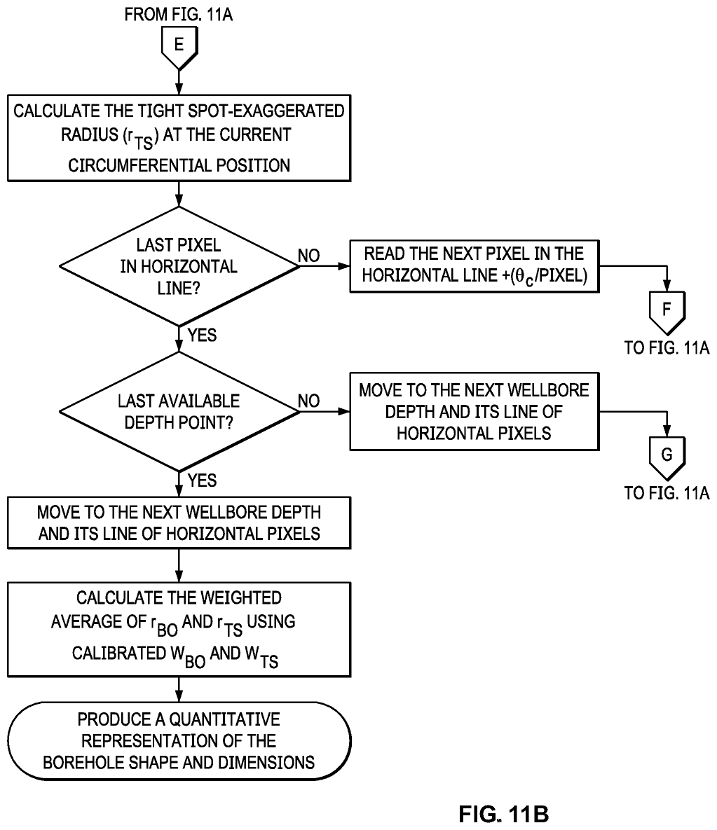

FIG. 11A and FIG. 11B illustrate an example flow chart 1100 of an algorithm for applying the calibrated ultrasonic image log model. In some implementations, once the calibration algorithm is applied to a certain imaging tool as described with reference to FIG. 10A and FIG. 10B, the image produced by this tool in new hole sections or new wells can be interpreted to produce a full 360° radial measurements by utilizing the algorithm described in FIG. 11A and FIG. 11B.

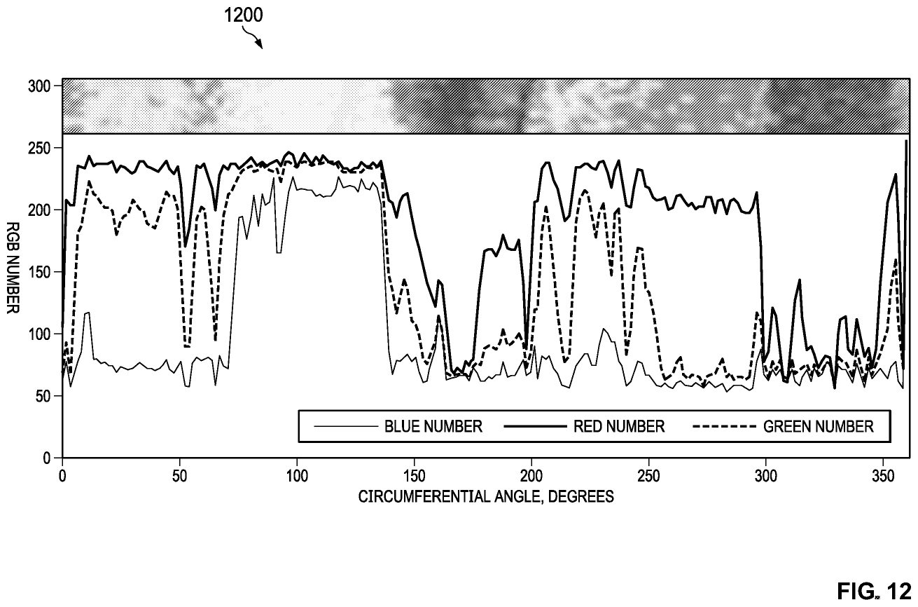

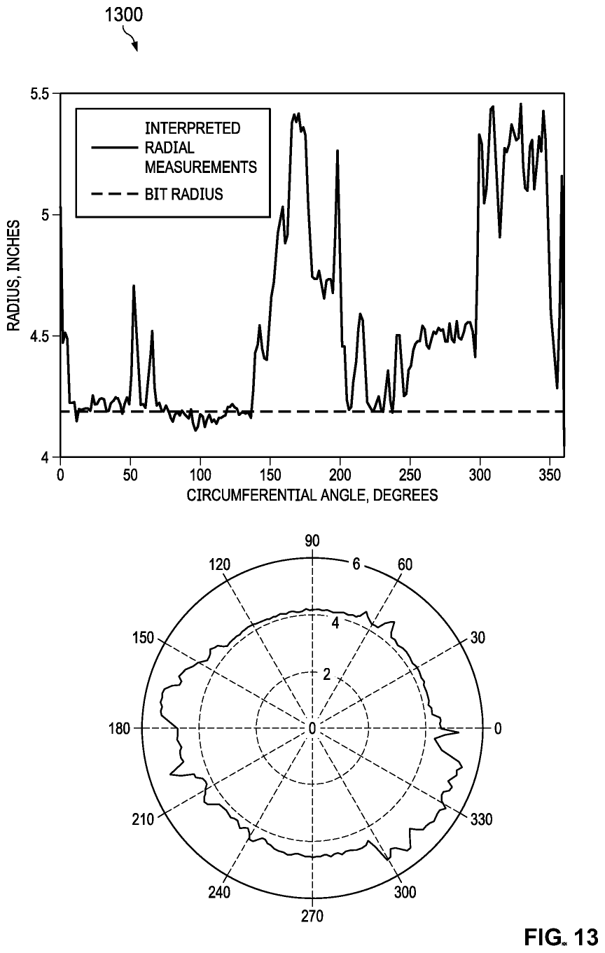

FIG. 12 and FIG. 13 illustrate the results of the interpretation of the ultrasonic image log in FIG. 9. FIG. 12 illustrates a plot 1200 of the correlation between the RGB number and the placement of enlargements and tight spots. This correlation can be used along with calibrating values from a mechanical multi-arm caliper log to calculate the radial measurements of the wellbore at different directions. In some implementations, the number of radial measurements produced is dependent on the number of pixels available in the image log (i.e. image resolution). In the example shown in FIG. 12, the image log has resolution of 221 pixels per the 360° degrees around the wellbore, which equates to about one radius measurement for each 1.63° degrees around the circumference of the wellbore. FIG. 13 illustrates a plot 1300 of the final results of the image log interpretations that are calibrated with mechanical caliper log readings in both Cartesian and polar coordinates. When comparing the ultrasonic image log interval in FIG. 9 to the interpreted radial measurements in FIG. 13, it can be seen that the dark regions in the image that signify the presence of wellbore enlargements are also reflected as radial measurements that are higher than the bit radius.

An example algorithm for field-based resistivity log imaging for borehole radial measurements is described next.

In some implementations, this algorithm converts this type of image logs into caliper readings that cover the 360° of the wellbore. The main difference between sonic-based images and resistivity-based images is that resistivity images normally contain several gaps due to the space between the electrical pads of the resistivity image logging tool. Resistivity based image logs also differ from sonic-based images in the way a breakout (or wellbore enlargement) can be identified. In sonic images, changes in color are sufficient, however, in resistivity images, changes in color alone can be misleading as they can signify laminations, faults, natural, and induced fractures along with breakouts. This means that resistivity logs are efficient indicators of ‘physical features’ within the wellbore, but they are poor indicators of the spatial (specifically, radial) extension of these physical features. Fortunately, there's one distinguishing feature of breakouts in resistivity logs which is the lowered resolution of the image at that breakout orientation. The lower resolution at breakouts is due to the electrical pad of the logging tool being further away from the wellbore wall when the resistivity image measurements are taken. This change in color resolution is exploited in this image interpretation algorithm to identify the orientation of breakouts, still, it's not sufficient to assess the extent of the enlargement. For this issue, provided multi-arm caliper readings are used to correlate the radial extent at every location in the wellbore. The pixels RGB readings are feature scaled using the following formula to yield the needed radii:

$$Scaled RGB = {CAL}_{min} + ( \frac{{RGB}_{adj} - {RGB}_{min}}{{RGB}_{max} - {RGB}_{min}} ( {CAL}_{max} - {CAL}_{min} ) ) ,$$

where CALₘᵢₙ and CALₘₐₓ are minimum and maximum multi-arm mechanical caliper radius readings respectively, which are used for calibration. RGB_{adj} is the adjusted RGB reading for filtering out noise and white gaps in the image. Using this method in interpreting image logs means that the availability of multi-arm caliper data is a necessity when resistivity images are used, which also means this analysis can't be done in real-time. This is to be expected since conventional resistivity-based image logs are not available through LWD in the same manner as density-based images.

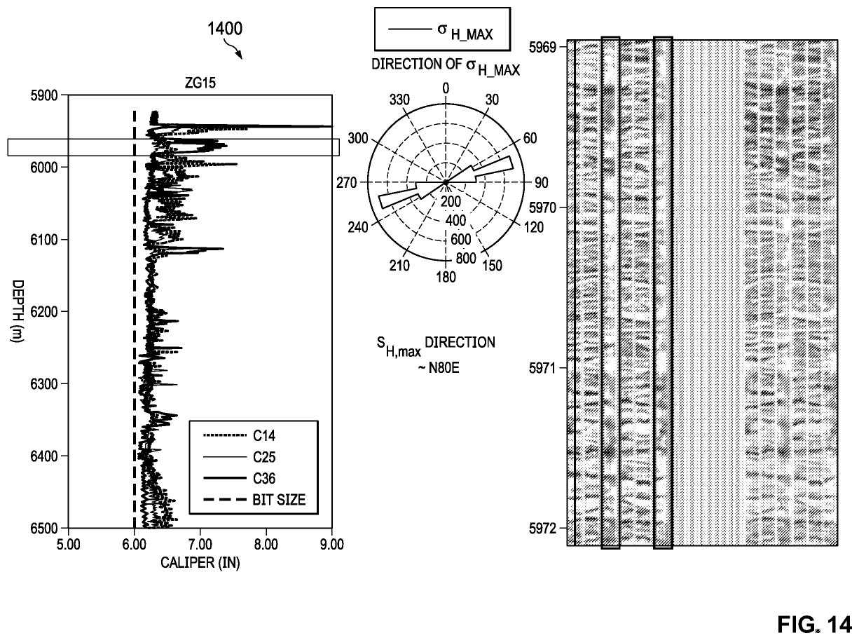

FIG. 14 illustrates an example plot 1400 of a resistivity image log along with mechanical caliper log readings. The regions highlighted in the two boxes in the image log to the right are the low-resolution areas that signify the presence of wellbore enlargements. The area highlighted with a box in the mechanical caliper log to the left is the interval from which the image log to the right was taken.

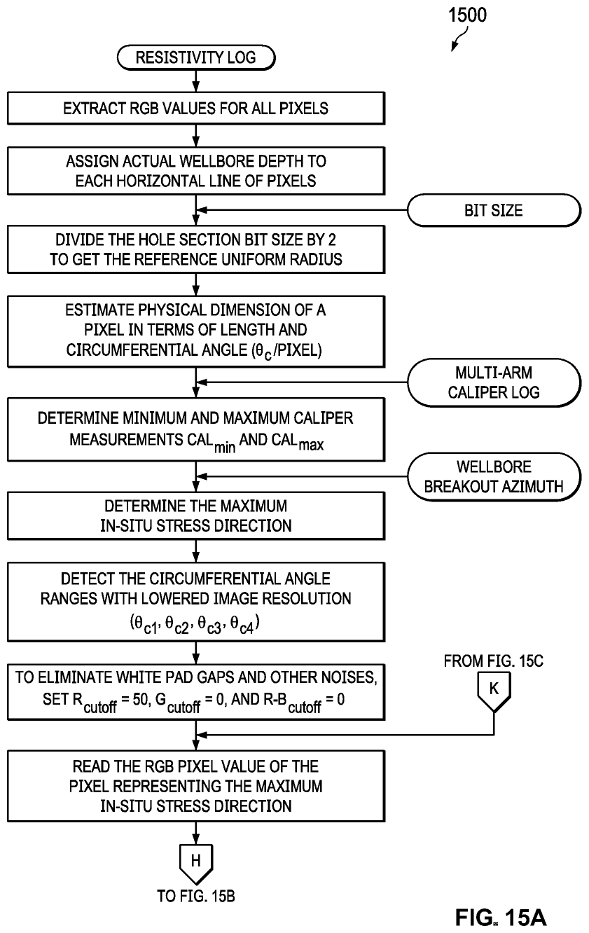

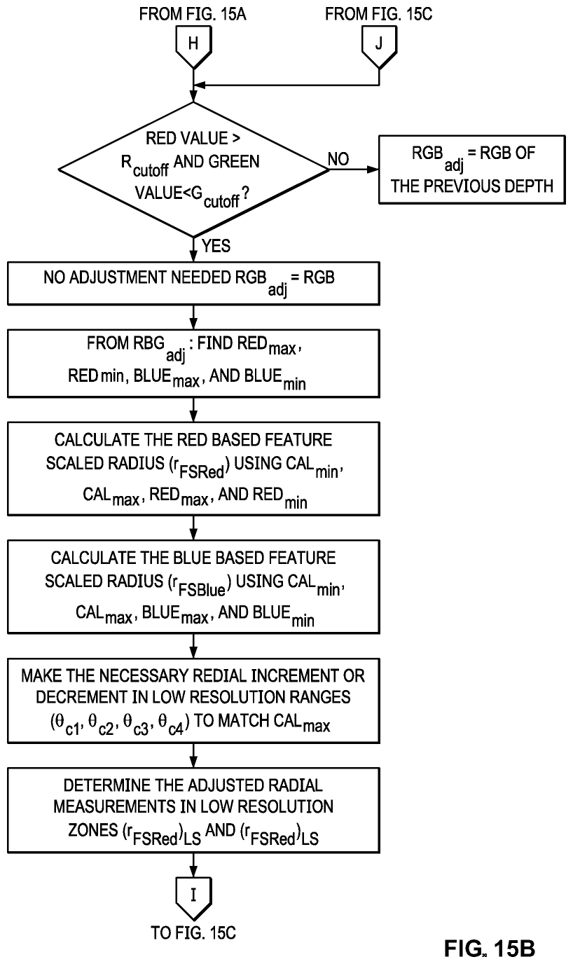

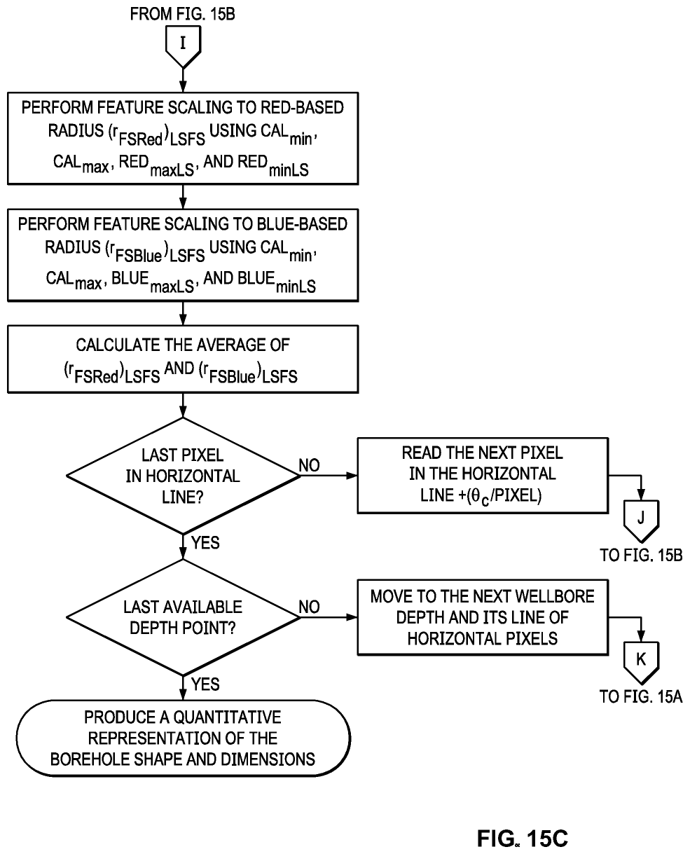

FIGS. 15A-15C illustrate an example flow chart 1500 of the algorithm for interpreting resistivity-based image logs. In some implementations, this algorithm uses data other than the image log itself to perform the interpretations process. These data can be the bit size, multi-arm mechanical caliper log measurements, and a definition of the direction of the maximum in-situ stress. The low-resolution zones can be defined using the circumferential angle ranges (θ_{c1}, θ_{c2}, θ_{c3}, θ_{c4}) where 1 and 2 define the starting and final angle of the first zone while 3 and 4 define the rage for the second zone. The RGB values can be adjusted to produce RGB_{adj} for the purpose of filtering out noise and white gaps in the image. This adjustment process can use an upper cutoff value of 0 for each pixel green color value (G_{cutoff}), a lower value of 50 for each pixel red color value (R_{cutoff}), and a lower value of 0 for the subtraction of blue from red color values (R−B_{cutoff}).

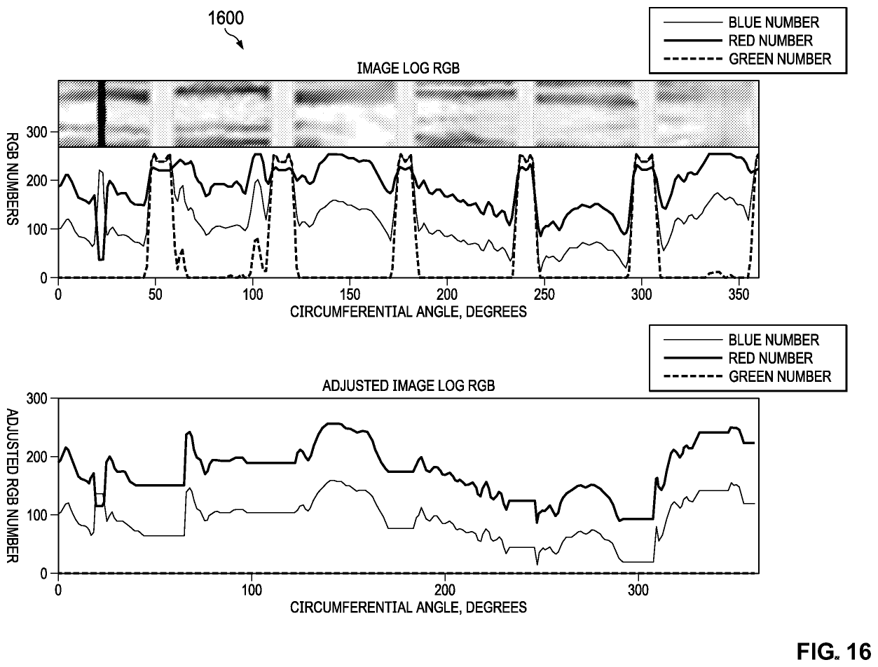

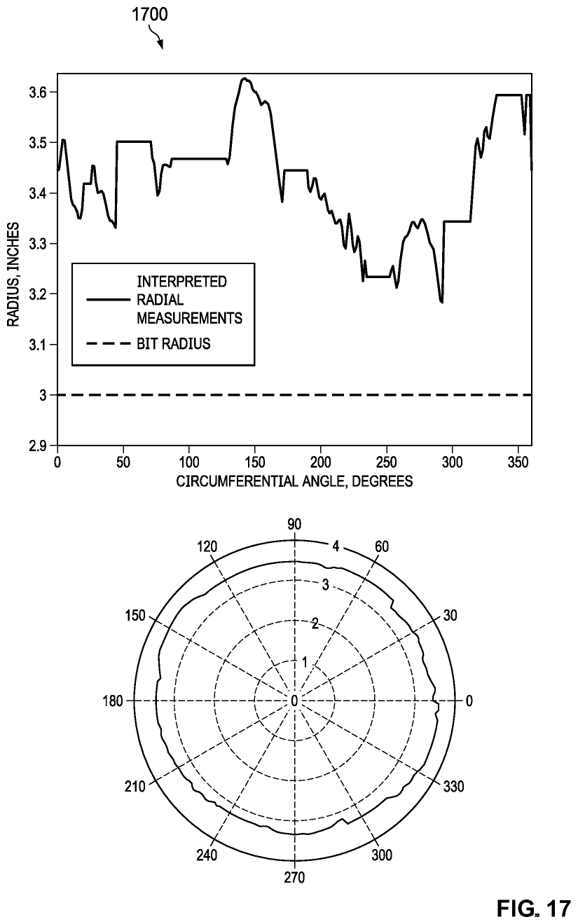

FIG. 16 illustrates a plot 1600 of the original RGB number of the resistivity image log from FIG. 14 and the adjusted RGB values to account for the image gaps and other image noise. FIG. 17 illustrates a plot 1700 of the final results of the image log interpretations that are calibrated with mechanical caliper log readings in both Cartesian and polar coordinates. When comparing the resistivity image log interval in FIG. 14 to the interpreted radial measurements in FIG. 17, it can be seen that the low resolution regions in the image that signify the presence of wellbore enlargements are also reflected as radial measurements that are higher than the bit radius. Also, when examining the mechanical caliper readings in the interval highlighted in red to the left in FIG. 14, it can be seen that there is an agreement that the wellbore is fully enlarged at different directions.

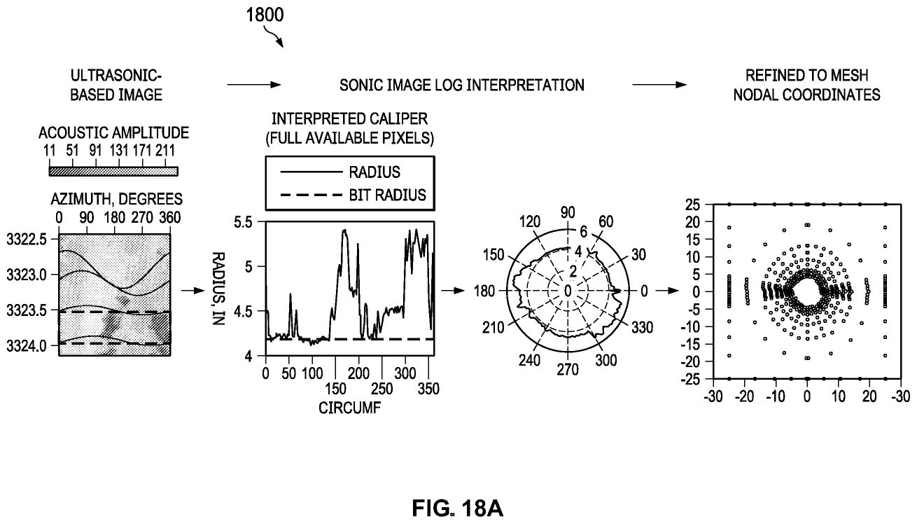

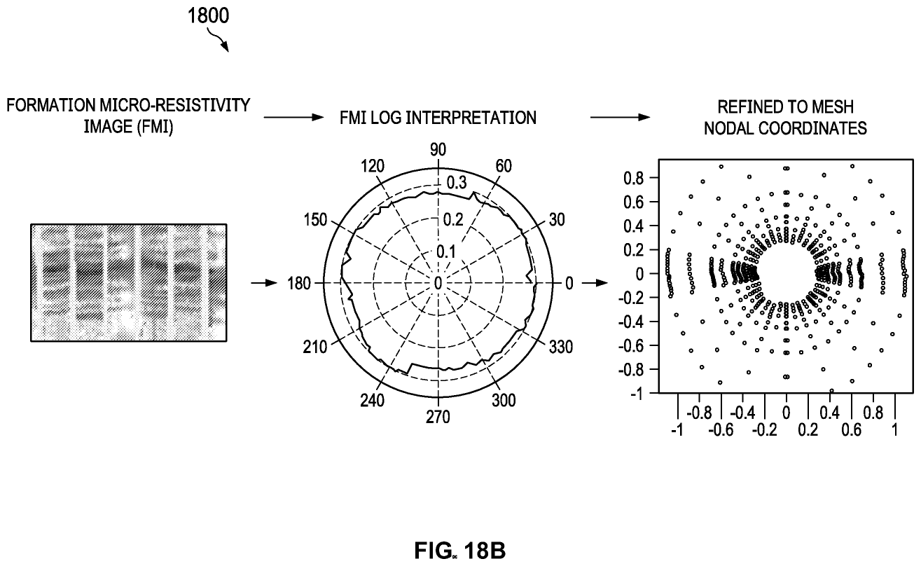

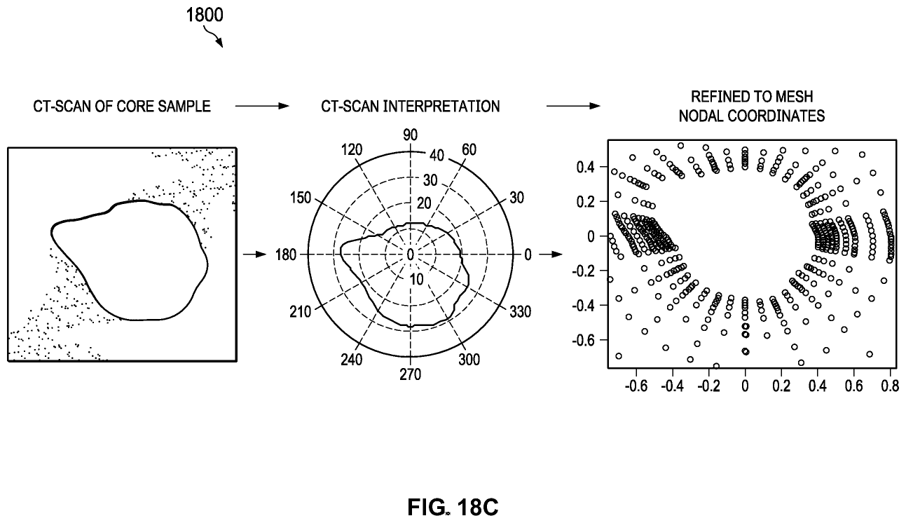

FIGS. 18A-18C illustrate an example process 1800 of converting the borehole shape interpretations from field and lab images into a three-dimensional finite element mesh nodal coordinate. In some implementations, once the radii measurements of a borehole are produced by the image analyzer algorithms described in the previous sections (for CT-scans, images, ultrasonic image logs, and resistivity image logs), the meshing function can receive these measurements and calculate the nodal coordinates of the mesh based on the measurement. In some examples, this is done by assigning a separate borehole radius measurement to each circumferential angle around the borehole. Then, the coordinates of each single line of nodes associated with each circumferential angle can be calculated with consideration of whether the line of nodes is placed at the center or at the boundary of an element in the mesh. This process is repeated for each radial line of nodes at all the specified vertical layers until the full coordinates describing the borehole or the wellbore and the rock formations the borehole penetrates is fully calculated. The meshing function can perform this process based on lab images, CT-scans, ultrasonic image logs, and resistivity image logs as shown in FIGS. 18A-18C.

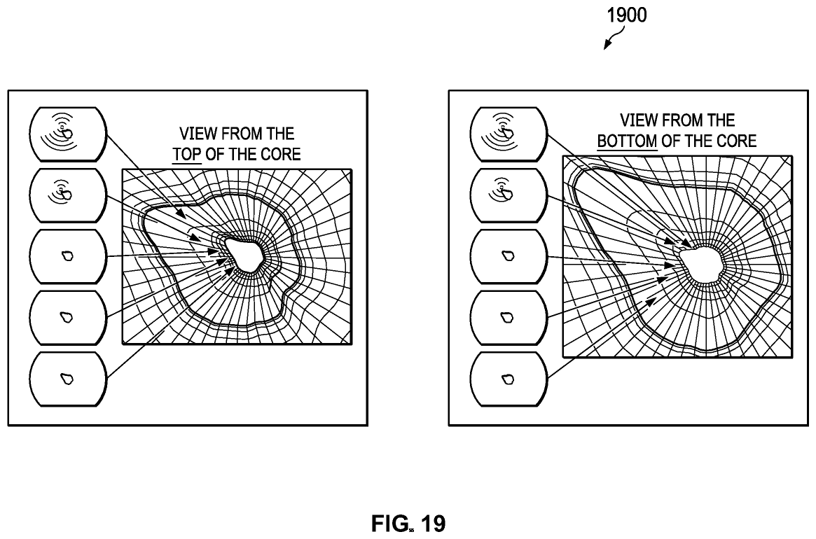

FIG. 19 is a plot 1900 of an example meshing function ability to reflect different borehole shapes based on CT-scan taken across different cross-sections of the core sample. For core samples, the mesh function reflects the different borehole shapes as described by CT-scans that are taken at different cross-sections of the core. The same goes for field-based images as the mesh can reflect the different shapes of the wellbore as described by the image log across different depths of the wellbore. An illustration of this function based on CT-scans is shown in FIG. 19.

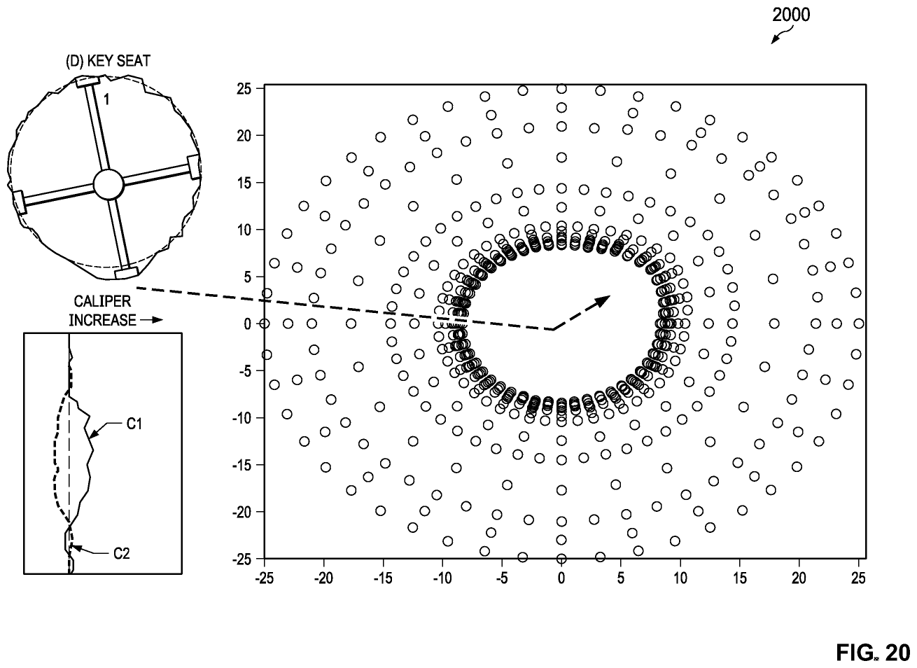

FIG. 20 is a plot 2000 of an example definition of key-seating and the results of the meshing function. In some implementations, this meshing function can also reflect different wellbore features, and this is specific to field-based (as opposed to lab-based) images. One wellbore feature can be key-seating, which is the enlargement of one side of the wellbore due to having the drillstring rest on that side. The meshing function can reflect the dimensions of the key-seat enlargement on the coordinate of the wellbore. An illustration of an example definition of key-seating and the results of the meshing function are shown in FIG. 20.

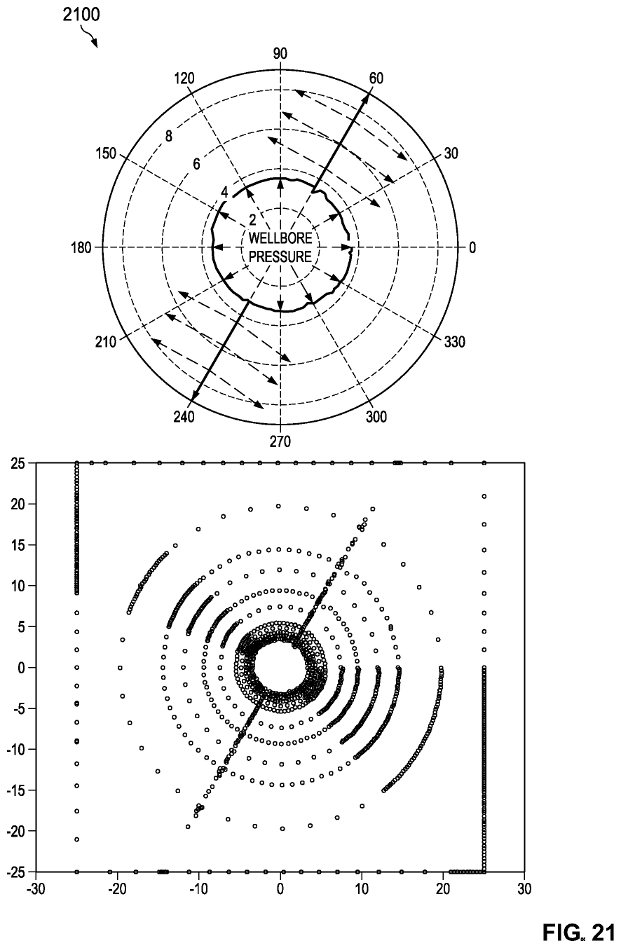

FIG. 21 is a plot 2100 of an example meshing of a static fracture. In some implementations, this meshing function can also reflect a fracture. The mesh function takes input such as the fracture aperture, length, tortuosity, and placement within the wellbore so that the geometry of the fracture can be reflected. Based on this, the mesh considers the fracture a part of the wellbore, which makes it exposed to the wellbore pressure. This way of meshing a fracture assumes that the fracture is a static feature of the wellbore. While this assumption may not be applicable for fracture propagation problems, it can be applied to other scenarios as it can be used to reflect the effect of drilling induced fractures, natural fractures, lost circulation, and wellbore strengthening on the drilling window. An illustration of an example of meshing of a static fracture is shown in FIG. 21, where the left plot shows the introduction of fractures to a non-uniform wellbore and a depiction of the wellbore pressure acting on both the wellbore wall and the fracture face, and the right plot shows the placement of nodal coordinate for the mesh of fractured non-uniform wellbore.

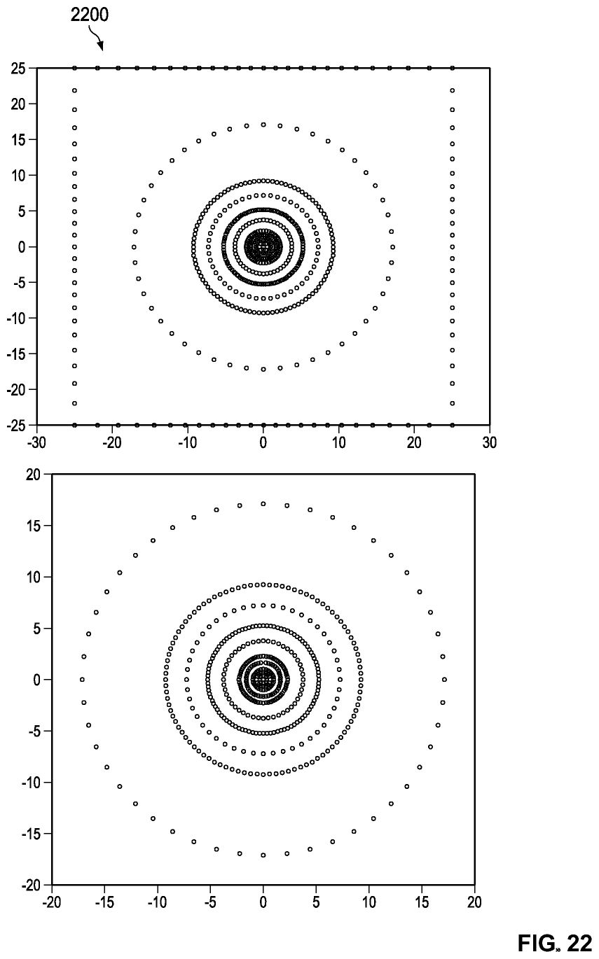

FIG. 22 is a plot 2200 of nodal coordinates of a mesh with finite boundary elements and a mesh with infinite boundary elements. In some implementations, this meshing function can also reflect the shape of the structure boundaries. When a mesh for a numerical model is created, the type of boundaries being used can be specified, and the type of boundaries being used can be reflected on the nodal coordinates calculated. The mesh function can consider a finite mesh boundary and an infinite mesh boundary. The finite mesh boundary means that the mesh elements at boundary of the structure, which will be subject to the outside stresses, are all fully formed elements. In one example, this fully formed element can be a 20-node brick element. As for the infinite mesh boundary, the mesh nodal coordinates are created for those that consider an infinite element, which can be a 20-node brick element with one of the cube faces removed resulting in a 12-node infinite element. The removed face signifies the infinite extension of the subterranean geologic formation being modeled. The mesh function can reflect this infinite nature of these elements by removing all nodes that would have described the removed face. The difference between these two boundaries as produced by the mesh function is shown in FIG. 22.

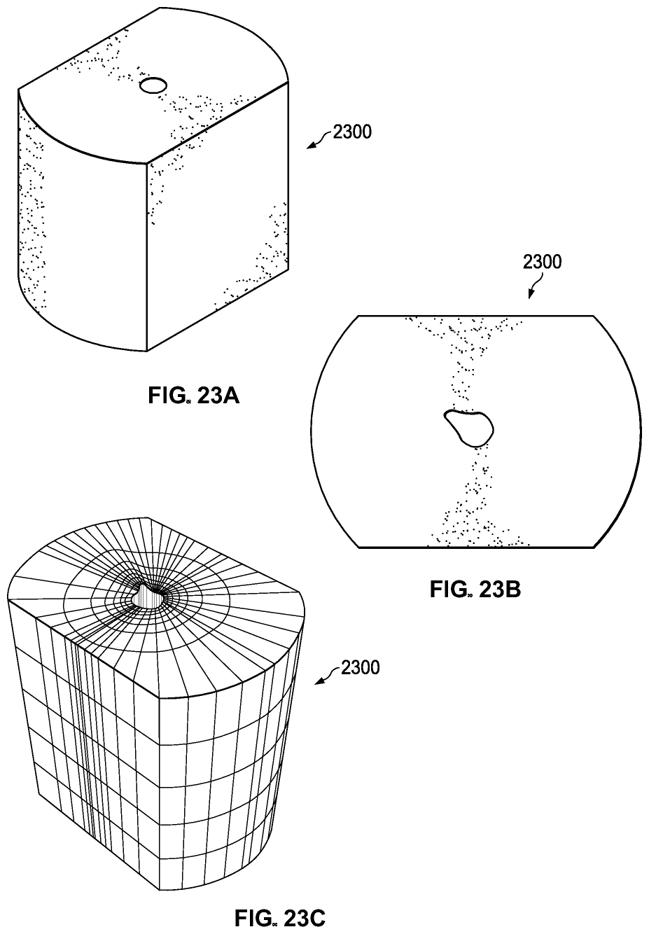

FIGS. 23A-23C are plots 2300 of an example mesh created for modelling core samples. In some implementations, the setup for the structure boundaries within the mesh can be altered when modelling core samples in experiments instead of wells in the field. A specialized meshing function can be created to model the core sample that are discussed with reference to the CT-scan image based algorithm. As these core samples have round boundaries on two sides and straight slabbed boundaries on the other two sides, the mesh function places finite element in all sides, but in a manner that mimics the core boundary shape.

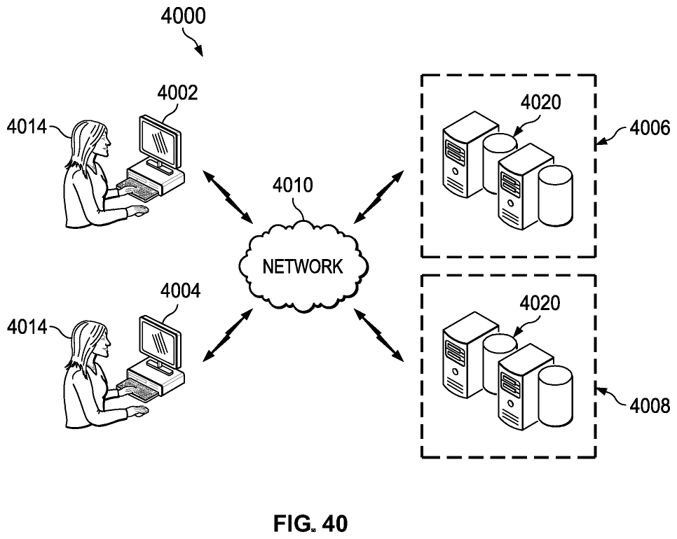

FIGS. 24A and 24B are plots 2400, 2450 of a three-dimensional finite element model 2402 for determining stresses in a formation surrounding a wellbore. The model 2402 includes a finite element mesh defined by one or more nodes and elements. Additional details of these finite element meshes and models are described in U.S. patent application Ser. No. 17/752,317 filed May 24, 2022 which is incorporated herein by reference in its entirety. In some examples, the finite element model 2402 represents a three-dimensional finite element model of the wellbore 102 and the formation 104 surrounding the wellbore 102. In some examples, the finite element model 2402 is a geomechanics model having an elasto-plastic solution capability. For example, the finite element model 2402 solves for elastic and plastic material behavior of rocks of the formation). The finite element model 2402 includes elements 2404 that include 20-node (quadratic) brick elements. The finite element model 2402 includes a physical model of overburden, under-burden, and the wellbore. A computer system (for example, the computer system 4000 described with reference to FIG. 40) creates the finite element model 2402, assigns loads, and assigns heterogeneous material properties to the finite elements 2404 as part of a pre-processing phase.

The computer system solves the finite element model 2402 to determine the cumulative influence of drilling mud weight variations, cyclic loads, drilling-induced wellbore enlargements, and production or depletion history to predict rock yielding and failure. The output of the finite element model 2402 is compared with reference data to determine the failure criterion that represents the rock failure.

The computer system solves the finite element model 2402 by minimizing the total potential energy of the finite element model 2402, which produces the following equilibrium condition:

u∫_{V}_{^{e}}((B^{T})D B)dΩ=∫_{V}_{^{e}}N^{T}F dΩ−∫_{S}_{^{e}}N^{T}TdΓ (1)

where u is the displacement, B and B^{T} are the strain-displacement matrix and its transpose, respectively. N T is the transpose of the quadratic Serendipity shape functions vector, which are derived for the 20-nodes isoparametric brick elements 2404 shown in FIG. 24B, D is the consistent tangent matrix, which is formulated based on the mechanical properties of the rock, F is the body force, and T is the traction force. The body and traction forces represent the in-situ stresses and mud weight loading on the wellbore. The integrations in equation (1) are performed over an element volume (V^{e}) with respect to the volume variable (Ω) or over an element surface (S^{e}) with respect to the area variable (Γ). The matrix resulting from the integral in the expression to the left is known as the stiffness matrix (K^{e}).

The finite element model 2402 uses a plastic flow rule for strain hardening to predict the plastic behavior of the rock, which occurs when the stresses are greater than the yield point. This means that the total strain is the addition of two components, which are poro-elastic strain (ε^{e}) and a plastic strain (ε^{p}). The plastic flow rule assumes that the flow direction is perpendicular to the yield surface and it is defined as:

$$Δ {ε}_{ij}^{p} = λ \frac{\partial ψ ( {σ}_{ij} )}{\partial {σ}_{ij}} \qquad ( 2 )$$

where εᵢⱼ^{p} is the plastic strain tensor, σᵢⱼ is the stress tensor, and λ is the plastic strain multiplier.

The associative flow rule is applied by assuming that the plastic potential surface is the same as the yield surface ψ. It also assumes the yield surface expands without changing the flow direction. The yield criterion is the Drucker-Prager criterion, where yielding will take place when the deviatoric stress tensor (Sᵢⱼ) and the mean stress (νₘ) satisfies the following relationship:

$$ψ ( {σ}_{ij} ) = \sqrt{\frac{1}{2} {S}_{ij} {S}_{ij}} - {a}_{0} + {a}_{1} {σ}_{m} = 0 \qquad ( 3 )$$

where constants α₀ and α₁ are determined experimentally as material properties and are used to correlate the Drucker-Prager criterion to the Mohr-Coulomb criterion.

The following expression for strain hardening is then used to calculate the scalar plastic strain ε^{p} from the plastic strain tensor determined by the flow rule:

$${ε}^{p} = \int \sqrt{\frac{2}{3} d {ε}_{ij}^{p} d {ε}_{ij}^{p}} \qquad ( 4 )$$

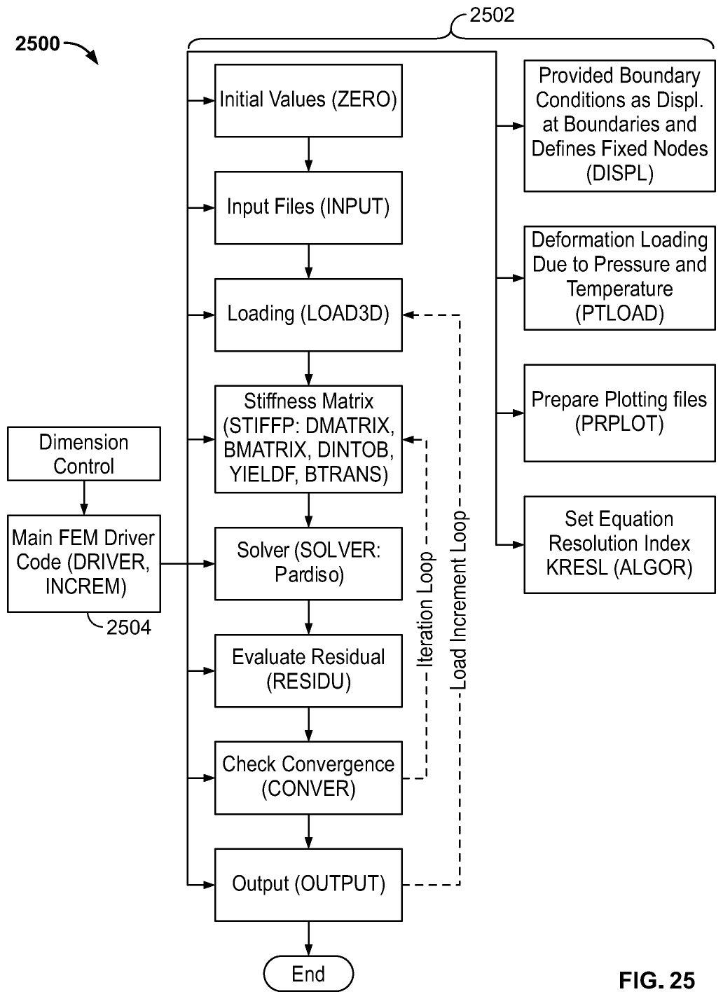

FIG. 25 is flow chart 2500 the solution process executed by the computer system to solve the finite element model 2402. The finite element model 2402 is solved using thirty-three subroutines and a driver code 2504, some of which are shown in FIG. 25. The driver code 2504 calls twelve main subroutines 2502 and these perform several functions including receiving the input file, applying loads to construct and assemble the global stiffness matrix, and solving the system of equations.

Upon solving the system of equations, as described by Equation (1), and determining the displacements u, the residual forces are calculated to check for convergence and equilibrium by subtracting the left-hand side from the right-hand side in the global form, where the left-hand side is the global stiffness matrix multiplied by displacement and the right-hand side is the body and traction forces. The value obtained from the subtraction of these two quantities should be equal to zero if the equilibrium condition is fully satisfied. However, that is not always achievable, therefore, a tolerance value is set to check for convergence. The tolerance value is usually set to be close but not equal to zero.

Once the residual forces are calculated and found to be less than the set tolerance value, convergence is said to be achieved, otherwise, the residual forces are carried to the next iteration. The same process is repeated for each separate load increment, where the load increments are defined in the input file manually. These processes are carried out in two loops with the convergence loop (or “iteration loop”) nested in the load increment loop as shown in FIG. 25. The computer system post-processes the finite element model 2402 after convergence is achieved.

The computer system uses one or more failure criterion models of rock and soil failure. For example, a Mohr-Coulomb criterion, a Mogi criterion, a Drucker-Prager criterion, and a Lade criterion can be used. In some examples, a Lade-adjusted Drucker-Prager criterion is used. In some examples, the models rely on lab testing to define the failure envelope of rocks. A failure envelope is defined by strength parameters which are specific to each developed model and are limited to certain limits of shear and normal stresses as observed in lab testing. These models suggest that rock failure in compression will take place if the stress state of the rock at the specific strength parameters is above the defined failure envelope.

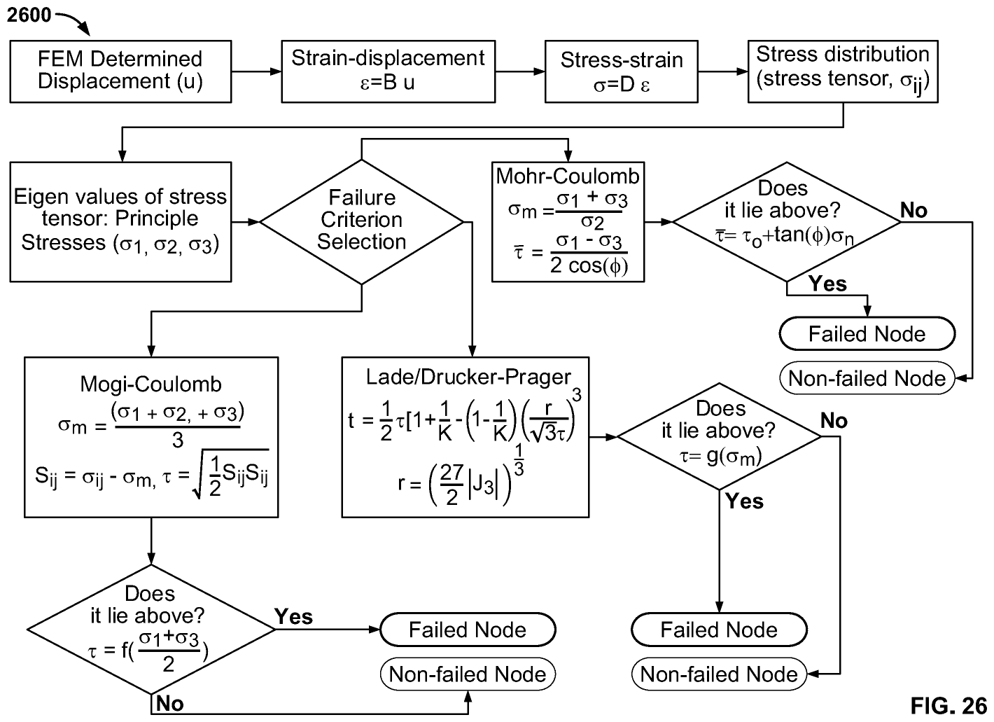

FIG. 26 is a flow chart 2600 of a method to determine whether nodes of an element of the finite element model have failed as part of a post-processing phase. The role of the failure criteria is to evaluate the stress state at each point (for example, at each integration point for each finite element 2404 of the finite element model 2402) against the strength parameters assigned to that particular integration point to determine whether that integration point has failed.

For example, if the computer system uses the Mogi failure criterion, the principle stresses calculated from the finite element model (ν₁, ν₂, σ₃) is used to determine the value of the octahedral shear stress (τ). Next, the failure criterion function (f) is calculated based on the strength parameters values assigned to each point. Finally, the value obtained from the failure criterion function (f) is subtracted from the calculated value of the octahedral shear stress (τ). If the computer system determines that the result of this subtraction is a positive value (meaning that this point lies above the failure envelope), the computer system determines that this point is predicted to fail.

An illustration of this process and the related expression for each conventional failure criterion are shown in FIG. 26. In some implementations, the computer system evaluates failure using multiple failure criteria to assess a best fitting one for each rock type. In all of the expressions in FIG. 26, the convention for compressive stress is negative.

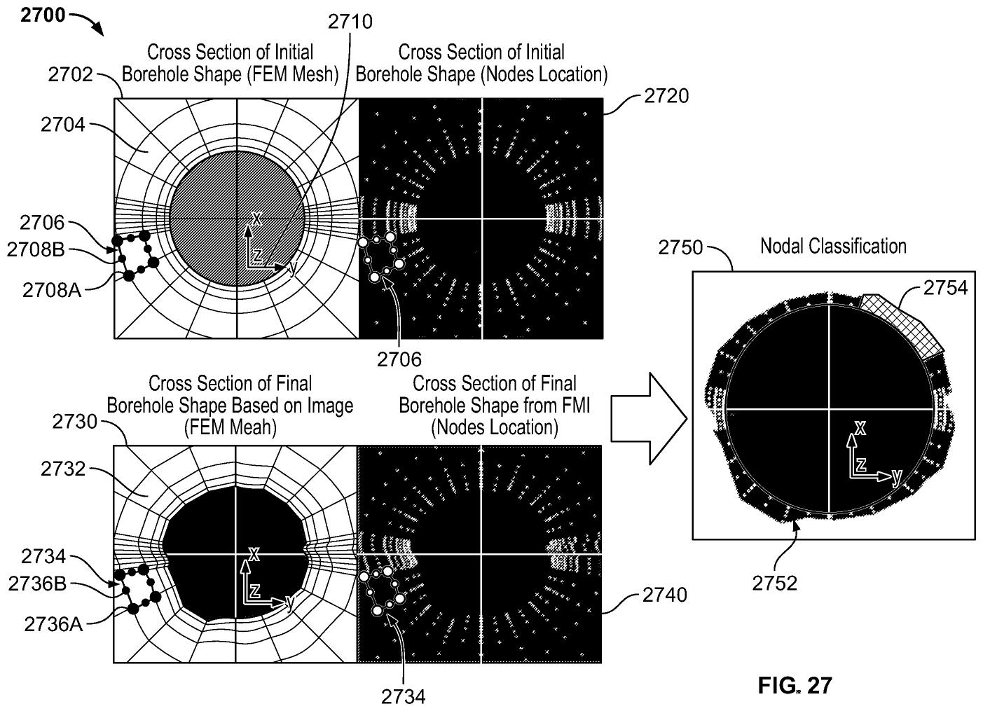

FIG. 27 is a plot 2700 illustrating a comparison process between an ideal mesh and a deformed mesh. Plot 2702 illustrates the ideal mesh 2704 of an ideal borehole (for example, a perfect cylindrical borehole) in a core sample. In some examples, the core sample shown in plot 2702 is the core sample 200 described with reference to FIG. 2. The ideal mesh 2704 includes many finite elements 2706, which are generally either linear elements (without mid-side nodes) or quadratic elements (with mid-side nodes). In some examples, the elements 2706 are the same as the elements 2404 described with reference to FIG. 24B. In this example, the ideal mesh 2704 includes quadratic elements 2706 with corner nodes 2708A and mid-side nodes 2708B (collectively referred to as “nodes 2708”). Each node 2708 has two dimensional (2D) or three-dimensional (3D) coordinates representing the location of the respective node relative to a coordinate system 2710. Plot 2720 illustrates the ideal mesh 2704 with the finite elements 2706 removed and the nodes 2708 shown. The computer system generates plot 2720 by looping over each finite element 2706 and plotting a marker (in this example, an ‘x’) at the 2D or 3D coordinates of each node 2708 of each respective finite element 2706. There are usually hundreds or thousands of finite elements 2706 in the mesh 2704.

Plot 2730 illustrates the deformed mesh 2732 of a deformed borehole in a core sample. In some implementation, the deformed mesh 2732 begins as the ideal mesh 2704 shown in plot 2702 and is deformed (or perturbed) based on reference data. In some examples, the reference data is lab-based images (for example, the CT scans such as those described with reference to FIGS. 8A-8B, DIC images, etc.). In some examples, the reference data is field-based data or images (for example, ultrasonic logs such as those described with reference to FIGS. 10A-10B and 11A-11B, resistivity logs such as those described with reference to FIGS. 15A-15C). The processes described with reference to those specific figures describe approaches to generate a finite element mesh based on CT scans, DIC images, ultrasonic logs, resistivity logs, and the like. In some examples, those processes start with an ideal mesh and perturb the finite element nodes so that the final shape of the mesh matches the images/data/logs. In some examples, those approaches generate a mesh that does not necessarily need to start with the ideal mesh 2704. In either of these cases, the computer system generates mesh 2732 which represents the deformed borehole shape (for example, after the borehole has been subject to loading conditions by the tri-axial testing machine or while the borehole is subject to down hole conditions in the field).

The deformed mesh 2732 includes many finite elements 2732. As noted the preceding paragraph, in some examples, the elements 2732 are the same as the element 2706 but are deformed or perturbed. The nodes 2736A, 2736B (collectively referred to as “nodes 2736”) of the deformed elements 2732 have spatial coordinates that represent the deformed location of the element 2732. This is in contrast to the ideal (or initial or original) location of the elements 2706 of the ideal mesh 2704. Plot 2740 illustrates the deformed mesh 2732 with the finite elements 2732 removed and the nodes 2736 shown. The computer system generates plot 2740 by looping over each finite element 2734 and plotting a marker (in this example, an ‘x’) at the 2D or 3D coordinates of each node 2736 of each respective finite element 2734.

In some implementations, the computer system determines a closed loop contour 2752 of the boundary of the borehole of the deformed mesh 2732 and superimposes the contour 2752 on a plot of the ideal nodal coordinates 2708. Plot 2750 shows an example of the contour 2752 superimposed on the ideal nodal coordinates 2708. The computer system performs a Boolean operation between the nodes 2708 and the contour 2752 to determine which nodes are in a failure region of the contour 2752 (for example, within the space between the contour 2752 and the ideal diameter of the borehole). In some examples, this space is referred to as a “failure region” of the core sample since material has failed in this region when comparing the ideal core sample to the deformed core sample. A portion of the failure region is represented by region 2754. The failure region 2754 generally has a diameter that varies around the entire circumference of the borehole and along the depth of the bore (for example, into and out of the page of the drawings).

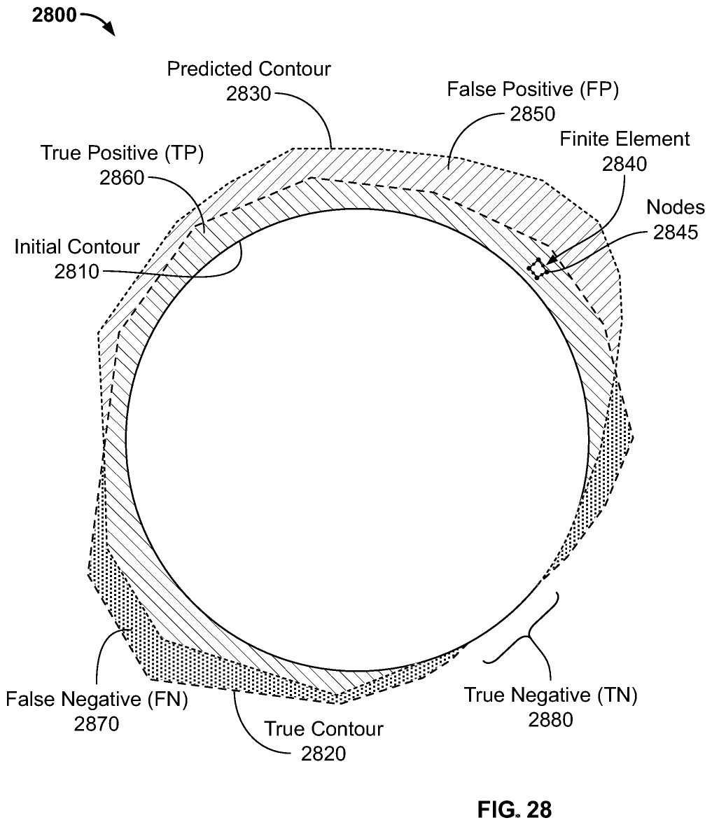

In the example described with reference to FIG. 27, an ideal mesh 2704 is compared to a deformed mesh 2732. In other examples, the ideal mesh 2704 is solved as part of a finite element model (for example, the finite element model 2402) and is subject to loading conditions representative of the tri-axial testing or of the downhole conditions of the wellbore. As described with reference to FIG. 26, the finite element model 2402 includes a failure criterion of the material that represents the rock of the core sample. The solved finite element model 2402 is post-processed to in the same manner as described above for the ideal mesh 2702 and compared to the deformed mesh 2732 to determine the nodal classification. This process is further described with reference to FIG. 28.

FIG. 28 is an illustration 2800 of a comparison among an initial contour 2810 of a borehole (for example, an initial or ideal shape of a borehole of the core sample 200), a predicted contour 2830 of the borehole as predicted by solving a finite element model (for example, the finite element model 2402), and a true contour 2820 as determined from reference data (for example, CT scans, DIC images, ultrasonic logs, resistivity logs, etc.). In this example, the ideal contour 2810 represents the contour of the borehole from the initial finite element mesh (for example, mesh 2704). The finite element model 2402 is solved with a particular failure criterion (for example, a Mohr-Coulomb criterion, a Mogi criterion, a Drucker-Prager criterion, and a Lade criterion) to model the failure of the rock of the core sample. The results of the solved finite element model 2402 are compared with “true” results from reference data (for example, as described with reference to FIG. 27).

The computer system assigns a confusion matrix parameter to each node 2845 of each finite element 2840 that represents the accuracy of the finite element model prediction. FIG. 28 shows four regions of the model that correspond to different confusion matrix parameters—false positive (FP) 2850 true positive (TP) 2860 false negative (FN) 2870 and true negative (TN) 2880. Table 1 indicates how the confusion matrix parameters are determined based on a relationship between the true results (for example, from the reference data) and the predicted results (for example, from the finite element model).

TABLE 1
Confusion matrix parameters.

| | | Predicted failure class from each | |
|---|---|---|---|
| | | failure criterion by solving | |
| | | the finite element model | |
| | | Non-failed | Failed |
| | | Node (−1) | Node (1) |
| True failure class from | Non-failed | True Negative | False Positive |
| reference data (for | Node (−1) | (TN) | (FP) |
| example, images) | Failed | False Negative | True Positive |
| | Node (1) | (FN) | (TP) |

Nodes that fall within the false positive (FP) 2850 region represent nodes that are predicted to fail based on the finite element model but have not failed in the actual reference data. Graphically, the nodes that fall within the false positive (FP) 2850 region are nodes that fall within the predicted contour 2830 and outside the true contour 2820. Nodes that fall within the true positive (TP) 2860 region represent nodes that are predicted to fail based on the finite element model and have failed in the actual reference data. Graphically, the nodes that fall within the true positive (TP) 2860 region are nodes that fall within the predicted contour 2830 and the true contour 2820 (for example, where the predicted contour 2830 and true contour 2820 overlap).

Nodes that fall within the false negative (FN) 2870 region represent nodes that are not predicted to fail based on the finite element model but have failed in the actual reference data. Graphically, the nodes that fall within the false negative (FN) 2870 region are nodes that fall within the true contour 2820 and outside the predicted contour 2830. Nodes that fall within the true negative (TN) 2880 region represent nodes that are not predicted to fail based on the finite element model and have not failed in the actual reference data. Graphically, the nodes that fall within the true negative (TN) 2880 region are nodes that where the true contour 2820 and the predicted contour 2830 is not defined (for example, the true contour 2820 and the predicted contour 2830 is along the borehole diameter or simply non-existent over a portion of the circumference of the borehole.

Once each node is assigned a confusion matrix parameter, the computer system processes the total number of nodes in each class to according to a statistical classification algorithm. In some implementations, the computer system determines one or more of the following parameters of a statistical classification:

$$Accuracy = \frac{TP + TN}{TP + FN + TN + FP}$$

$$Recall ( True Positive Rate or TPR ) = \frac{TP}{TP + FN}$$

$$Specificity ( True Negative Rate or TNR ) = \frac{TN}{TN + FP}$$

$$False Positive Rate ( FPR ) = \frac{FP}{FP + TN}$$

$$Precision ( Positive Predictive Value , PPV ) = \frac{TP}{TP + FP}$$

$$F 1 \_Score = 2 \times \frac{Precision \times Recall}{Precision + Recall}$$

In the above equations, “TP”, “TN”, “FN”, “FP” represent the total number of nodes that have the true positive, true negative, false negative, and false positive classification, respectively. In some examples, “Accuracy” represents an overall ratio of mesh nodes where failure was accurately predicted. Preferably, accuracy should be maximized. “Recall” represents a measure of the ability to predict failed mesh nodes correctly. Preferably, recall should be maximized. “Specificity” represents a measure of the ability to predict non-failed mesh nodes correctly. Preferably, specificity should be maximized. “False positive rate (FPR)” represents the ratio of non-failed mesh nodes that were predicted incorrectly. Preferably, the false positive rate should be minimized. “Precision” represents the ratio of the failed mesh nodes that were predicted correctly. Preferably, precision should be minimized. “F1-score” represents an overall measure of the predictive ability of the finite element model using the particular failure criterion. The F1-score is different than accuracy because it is not affected by the skewness of the results towards a certain class.

### Example #1: Core Sample with Lab Reference Data

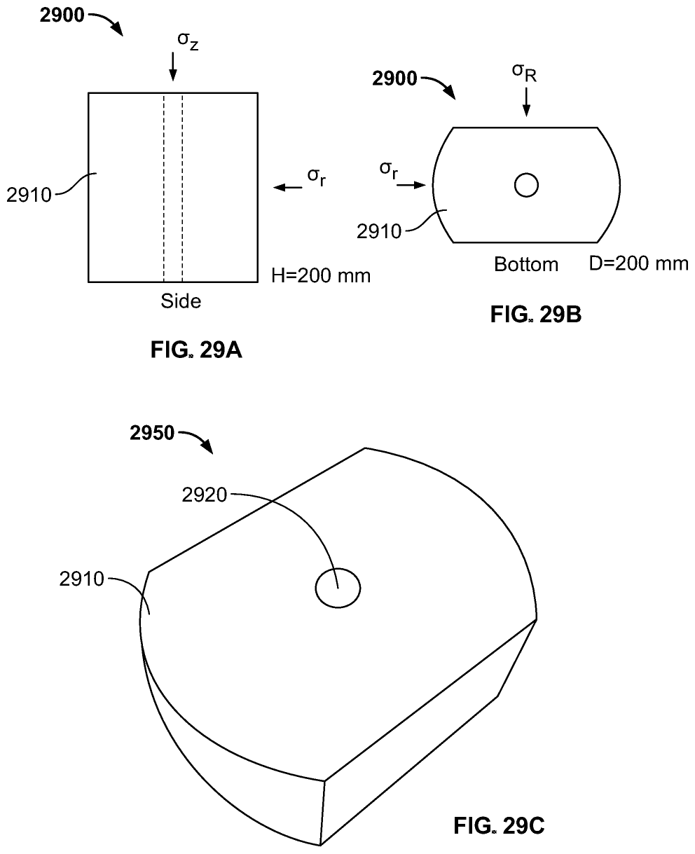

FIGS. 29A-29B are schematics 2900 of a core sample 2910 subject to tri-axial compression. FIG. 29C is an image 2950 of the example core sample 2910. The core sample 2910 includes a borehole 2920. In this example, the core sample 2910 is dimensioned and shaped to fit within a tri-axial testing machine so that tri-axial stresses can be applied to the core sample 2910. The core sample 2910 is formed of Castelgate sandstone. The core sample 2910 has a length and width of around 7.87 inches (200 mm), a slabbed outer diameter of around 5.50 inches (140 mm), and a borehole 2920 with a diameter of around 0.87 inches (20 mm). The stresses (σ_{z}, σᵣ, σ_{R}) are applied in different combinations in the three directions as shown in FIGS. 29A and 29B. The stress σ_{R} is the maximum horizontal stress (perpendicular to the borehole axis), which is applied in direction of the slabbed face of the core sample 2910.

Table 2 indicates details from five different tests that were performed using the approach described in this disclosure. The results illustrate the predictive capabilities of the finite element model using various failure criteria. These experiments also use high resolution CT-scans to evaluate the borehole failure in the post-test core samples.

TABLE 2
Stress combinations and sample dimensions of the true-tri-axial tests.

| | | | | Internal | External | Slabbed |
|---|---|---|---|---|---|---|
| Experiment | K_{z} = | Kᵣ = | Length, | Diameter, | Diameter, | Length, |
| Number | σ_{z}/σ_{R} | σᵣ/σ_{R} | mm | mm | mm | mm |
| EXP-1 | 1 | 1 | 200.05 | 19.83 | 199.8 | 138.55 |
| EXP-2 | 1 | 1/3 | 199.90 | 19.9 | 199.55 | 138.49 |
| EXP-3 | 2/3 | 2/3 | 199.86 | 19.88 | 199.7 | 138.82 |
| EXP-5 | 2/3 | 2/3 | 199.74 | 20.0 | 199.85 | 138.32 |
| EXP-6 | 2 | 2/3 | 200.05 | 19.88 | 199.7 | 138.82 |

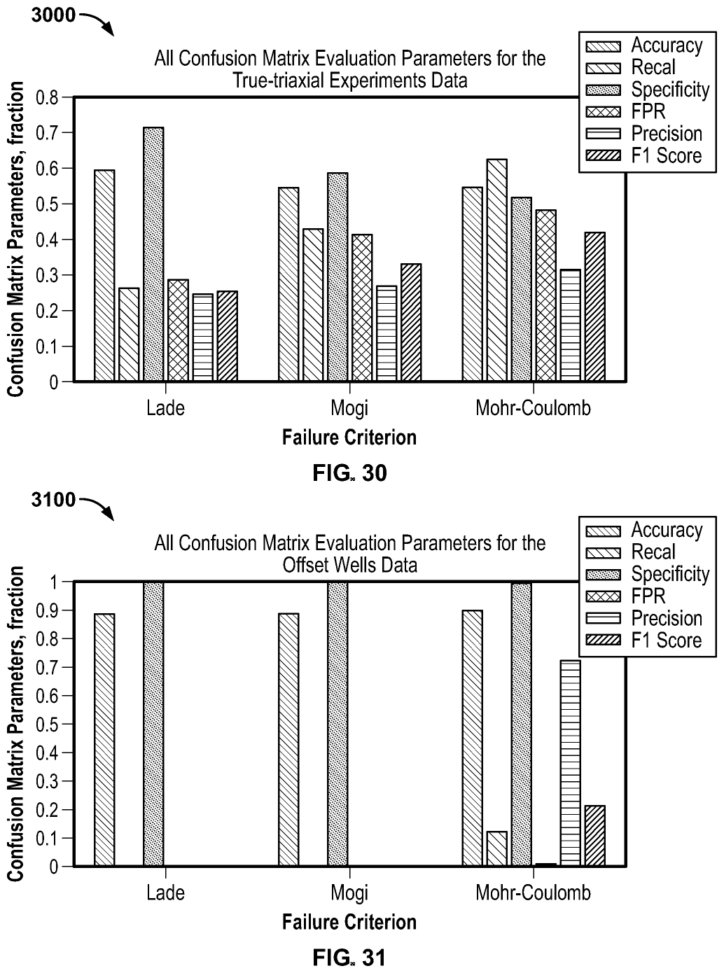

Each of these five experiments are modeled to generate a 3D finite element mesh and processed using the CT-scan interpretation algorithm as described with reference to FIGS. 8A-8B to generate a true contour (for example, similar to the true contour 2820 described with reference to FIG. 28). The size of the cumulative dataset across all five experiments is 59,280 data points (or nodes). The performance of each failure criterion (in this example, Mohr-Coulomb, Mogi, and Lade) is evaluated at each of these 59,280 data points (or nodes) by solving a separate finite element model of the core sample for each failure criterion. The results of this evaluation using the confusing matrix parameters is shown in FIG. 30.

FIG. 30 is a plot 3000 of confusion matrix evaluations of the statistical classification for each of three failure criteria based on core sample imaging reference data. In this example, the three failure criterion are the Mohr-Coulomb criterion, the Mogi criterion, and the Lade criterion. The core sample imaging reference data represents CT scans acquired while the core sample is subject to tri-axial experiments using a tri-axial testing machine. The computer system generates plot 3000 and determines that the Mohr-Coulomb produced the best overall accuracy based on its F1-Score, which evaluates the overall performance for both the failed and non-failed nodes. In some implementations, the computer system determines the failure criterion by selecting the failure criterion associated with the largest F1-Score among all the failure criteria.

### Example #2: Logging Data from a Wellbore

The sourcing and processing of relevant field offset wells data is illustrated in this example. The rock surrounding the well is sandstone. In this example, reference data is collected using logging device at five offset wells. The first type of field data acquired from the offset wells is the profile of the well. The profile data includes the wellbore size, inclination, and azimuth. While most offset wells are vertical, it is not uncommon for vertically planned wells to experience some degree of unintentional deviation while drilling. It is important that these deviations are included in the wellbore orientation surveys and reflected in the reference data from the offset wells. The second type of data to be acquired are the in-situ stress values and the definition of intervals where a representative image log is available. For the in-situ stress, the minimum horizontal stress values are gathered based on leak-off tests or mini-frac tests and the vertical overburden stress values are based on density log measurements. The maximum horizontal stress values and directions are determined based on image log analysis and stress polygon plots. The interval of a representative image log is the depth that has an image log of acceptable image resolution that exhibits wellbore failure that is representative of the failure throughout the wellbore section. In this example, all the image logs are resistivity-based and are acquired by a formation micro-imager or micro-scanner (FMI/FMS) tool.

The values of the in-situ stress and their directions along with the wellbore profile information such as inclination and azimuth are used by the computer system to determine the loading conditions for the finite element model. The predicted unconfined compressive strength (UCS) values are used for interpolating the stress-strain curve for constitutional modelling. For example, in order to have an estimation of the rock's intrinsic mechanical, strength, and failure properties, a tri-axial test of a core sample of this rock is conducted. Stress-strain curves are the raw output from this experiment. The stress-strain curves are used to calculate the UCS values. Since the drilling process penetrates through different rock types and heterogeneous formations, this experiment is preferably conducted for each rock type and for each range or UCS value of the same rock type. This is background from the lab/experimental work side. When it comes to the actual drilling operation, the systems and methods acquire continuous measurements from the wellbore as the wellbore is drilled. These measurements are produced by the logging tools. These measurements are used by the systems to estimate and/or predict (for example, rather than measure) UCS values. Based on these predictions, as shown in the 8th column of Table 3, the systems assign and/or interpolate a stress-strain curve to each depth point based on its predicted UCS value.

TABLE 3
Details of offset wells profiles, training and testing classification, in-situ
stress values and their directions, the definition of intervals where a representative
image log is available, and the predicted UCS for all the available offset wells.

| | | | | Maximum | | | | |
|---|---|---|---|---|---|---|---|---|
| | | | | Horizontal Stress | Minimum | | | |
| | | | Vertical | σ_{H} and | Horizontal | Depth of a | Predicted | Wellbore |
| | Inclination, | Azimuth, | Stress | its direction, | Stress σₕ, | Representative | UCS, | Diameter, |
| Well | degrees | degrees | σ_{V}, ppg | ppg and degrees | ppg | FMI, ft | psia | inches |
| Well-1 | 0° | 0° | 17.4 | 21.3, 42°N | 12.65 | 13,337 | 9,000 | 8.5 |
| Well-2 | 7° | 234° | 18 | 20.8, 56°N | 12.1 | 12,795 | 9,000 | 6 |
| Well-3 | 9.6° | 275° | 18.6 | 21.6, 57°N | 12.2 | 12,909 | 8,000 | 8.5 |
| Well-4 | 5° | 98° | 17.9 | 20.5, 32°N | 12.38 | 12,790 | 9,000 | 8.5 |
| Well-5 | 1° | 87° | 18 | 20.9, 59°N | 12.2 | 12,994 | 9,000 | 8.5 |

An engineer modeled all the offset wells using finite element models and processed them using the resistivity image log interpretation algorithm as described with reference to FIGS. 15A-15C. In this example, like example #1, the size of the cumulative dataset is 54,096 data points (or nodes). The accuracy of each failure criterion is evaluated at each of these data points. The results of this evaluation using the confusing matrix parameters are shown in FIG. 31.

FIG. 31 is a plot 3100 of confusion matrix evaluations for three different failure criteria based on wellbore logging data (field data). In this example, the three failure criterion are the Mohr-Coulomb criterion, the Mogi criterion, and the Lade criterion. The wellbore logging data or field data represents resistivity-based measurements acquired by a formation micro-imager or micro-scanner (FMI/FMS) tool. The computer system generates plot 3100 and determines that the Mohr-Coulomb produced the best overall accuracy based on its F1-Score, which evaluates the overall performance for both the failed and non-failed nodes. In some implementations, the computer system determines the failure criterion by selecting the failure criterion associated with the largest F1-Score among all the failure criteria.

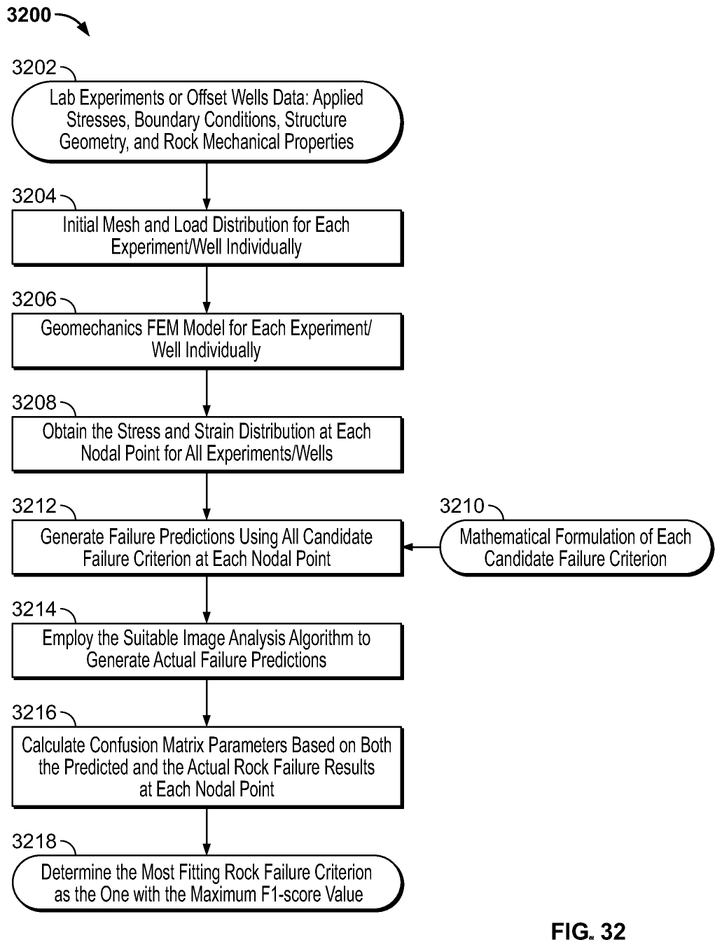

FIG. 32 is a flowchart 3200 illustrating an example process for determining the failure criterion based on a maximum F1-score value. In some implementations, the process is implemented by a computer system such as computer system 4000 described with reference to FIG. 40. At step 3202, lab experiments are conducted and/or offset well data is acquired so that enough information is generated to develop a finite element model. In some examples, this reference data includes applied stresses, boundary conditions, structure geometry, and rock mechanical properties.

At step 3204, an initial mesh and load distribution for each experiment (for example, each core sample) and/or well (for example, each well of the offset wells) is generated. At step 3206, a geomechanics finite element model is developed for each experiment and/or each well. In some examples, the finite element model is the same as or similar to the finite element model 2402 described with references to FIGS. 24A and 24B. At step 3208, the computer system solves the finite element model and obtains stress and strain distributions at each nodal point (or node) for each experiment and/or well.

At step 3210 the failure criteria are established. In some examples, the failure criterion include a Mohr-Coulomb criterion, a Mogi criterion, a Drucker-Prager criterion, a Lade criterion, as described with reference to FIG. 26. Other failure criteria can also be used. At step 3212, the computer system generates failure predictions using all of these failure criteria at each nodal point (or node) by solving separate finite element models for each failure criteria.

At step 3214, the computer system implements an imaging analysis algorithm to generate actual failure predictions. In some examples, this results in the true contour 2820 described with reference to FIG. 28 and the deformed mesh 2732 described with reference to FIG. 27. In some examples, the imaging analysis algorithm is the CT scan algorithm 800 described with reference to FIGS. 8A and 8B, the ultrasonic log algorithm 1000, 1100 described with reference to FIGS. 10A, 10B, 11A, and 11B, and/or the resistivity log algorithm 1500 described with reference to FIGS. 15A-15C.

At step 3216, the computer system calculates confusion matrix parameters based on both the predicted and actual rock failure results at each nodal point (or node). In some examples, this results in classifying each node as one of false positive (FP), true positive (TP), false negative (FN), or true negative (TN) as described with reference to FIG. 28 and Table 1. At step 3218, the computer system determines the failure criterion by selecting the failure criterion associated with the largest F1-score using a statistical analysis as described with reference to FIGS. 30 and 31.

Example applications of the aforementioned numerical models are described next.

In some implementations, one application of the aforementioned numerical models is providing an advisory on the drilling window limits. The numerical model, which is setup based on the image algorithms and the meshing function described in this disclosure, can provide recommendations of the maximum and minimum allowable mud weight for safe and optimal operations. These mud weights can be determined for the purpose of avoiding wellbore rock failure. Although drilling window advisory systems are common in the industry, some are limited as they produce static recommendations or pre-drilling recommendations. Some existing models lack the ability to update drilling window recommendations in response to commonly occurring drilling events in a dynamic and deterministic manner. The numerical models supported by the image algorithms and the meshing function described in this disclosure can consider the influence of commonly occurring drilling events through the input provided from the shale shakers analysis.

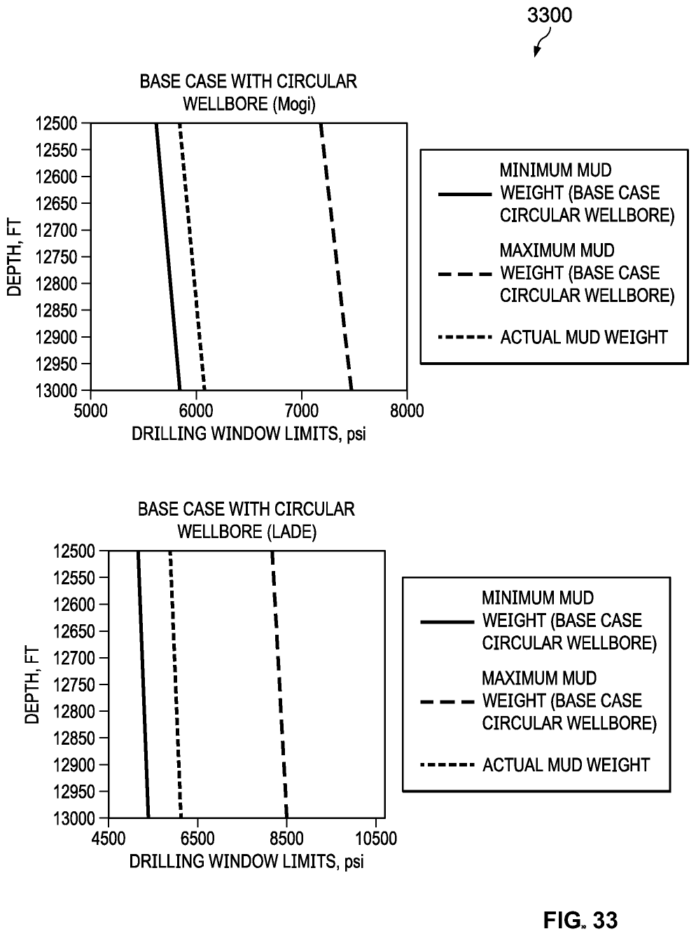

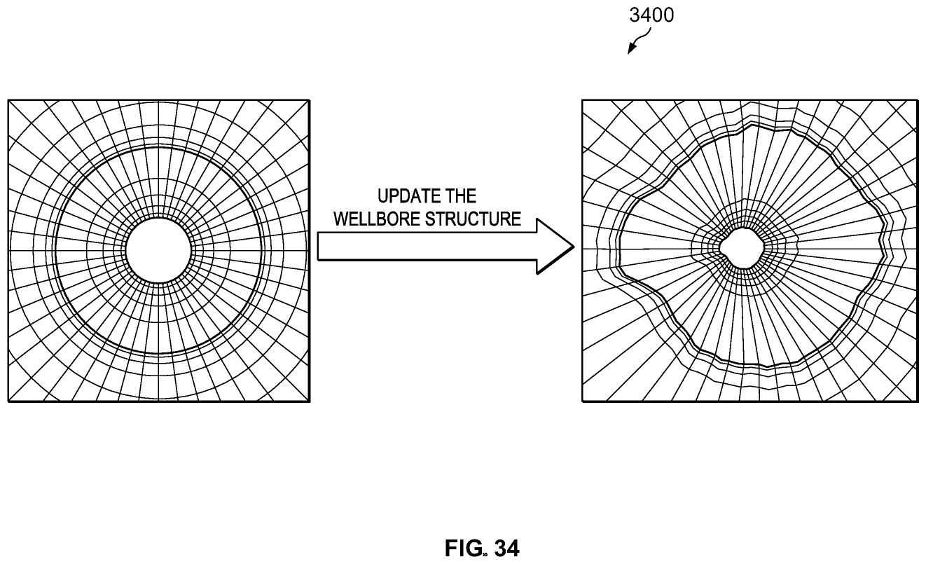

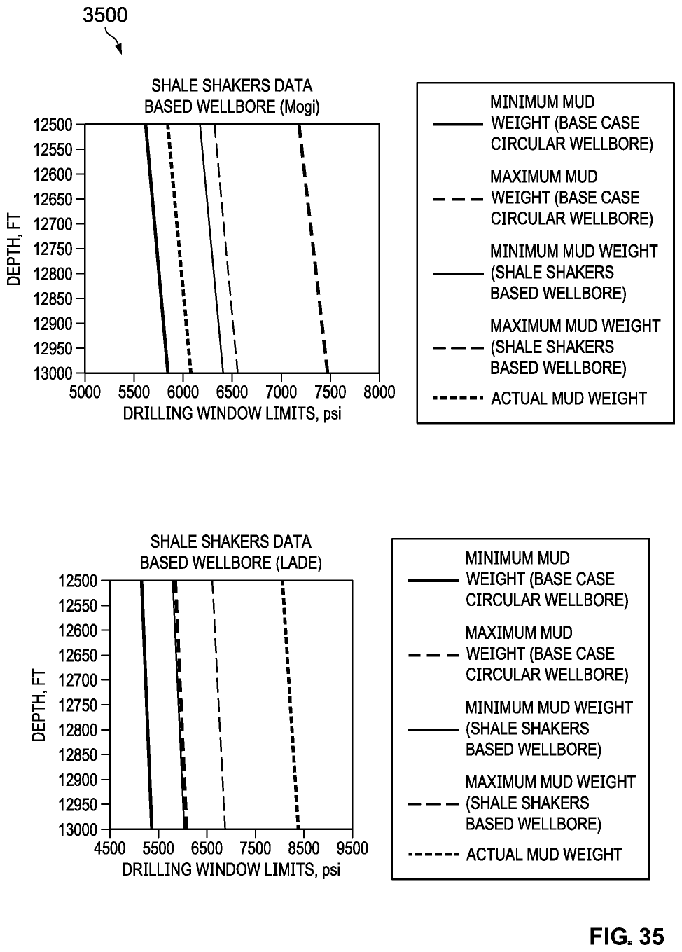

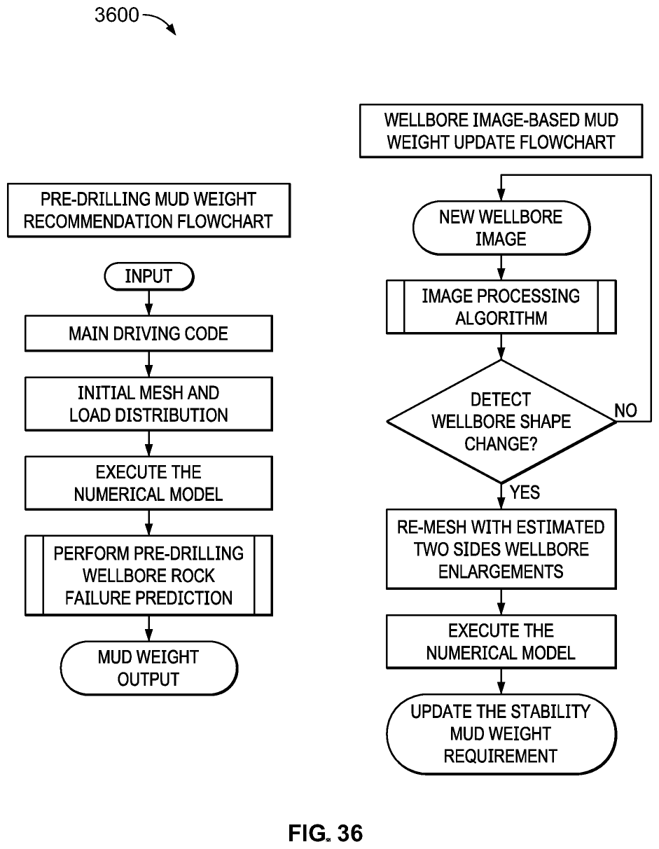

FIG. 33 illustrates example pre-drilling recommendations for the drilling window. In some implementations, the drilling window recommendation can be updated based on the input from logging while drilling wellbore images or from the shale shakers image analysis. Through this analysis, the wellbore structure mesh can be updated as shown in FIG. 34. This updated mesh can be inserted into the numerical model described in this disclosure for the purpose of providing the new mud weights limits or downhole pressure required for preventing wellbore failure. An example of the updated output of the model, which relates to recommending mud weights for preventing wellbore rock failure is shown in FIG. 35. Flow charts depicting the distinction between the two processes to determine the pre-drilling mud weight and the while-drilling mud weight update are shown in FIG. 36.

In some implementations, another application of the aforementioned numerical models is improving the accuracy of borehole rock failure prediction. The image analysis algorithms described in this disclosure can be used to overcome simplifying assumptions in some existing drilling geomechanics modeling, and consequently to improve rock failure prediction beyond the use of the conventional failure criteria such as the Mohr-Coulomb, Mogi, and Lade criterion. The use of image analysis algorithms has an advantage over traditional methods, as the use of image logs analysis and re-meshing functions can enable the reflection of the actual shape of the wellbore on the numerical model mesh. Additionally, rather than relying on generalized and conventional failure criteria for predicting rock failure spatially around the wellbore, the numerical model described in this disclosure can use a statistical or a machine learning algorithm that is trained based on the outcome of borehole image logs analysis and the output of the numerical model. The image analysis can set the limits, distribution, and extent of boreholes rock failure for the machine learning algorithm, based on the failure outcome from previous wells or lab experiments.

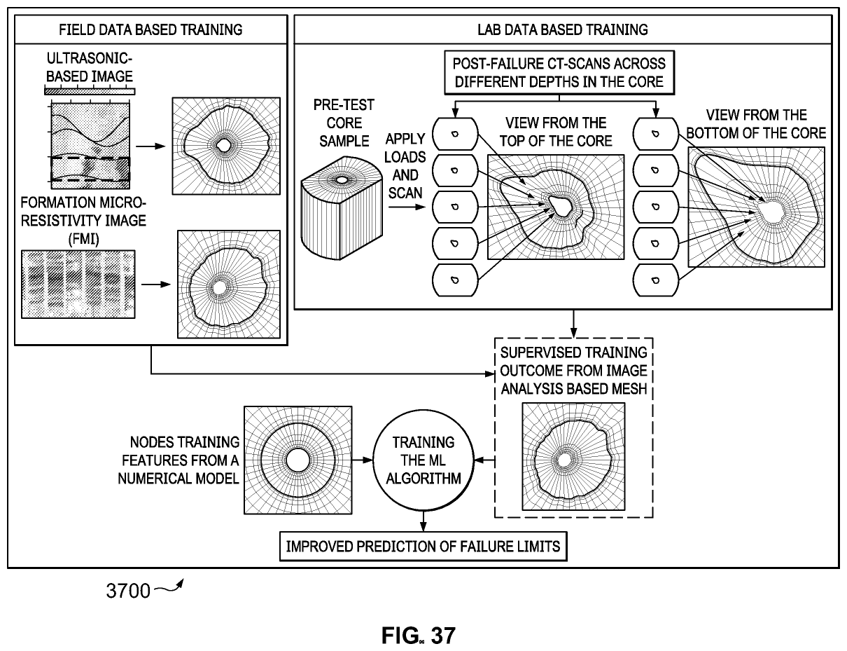

In some implementations, the supervised training process of the machine learning algorithm can be performed as shown in FIG. 37 using the outcome of the image analysis algorithms. Based on this illustration, relevant information from offset wells can be gathered, which can then be used to model the circular (pre-failure) wellbore in the numerical model as shown in the left-bottom corner in FIG. 37. This can yield the stress distribution at each node in the mesh in the left-bottom corner, which can be represented by different stress invariants and scalar plastic strain. Consequently, this stress distribution can be used to generate an initial prediction of failure based on one of the aforementioned conventional failure criteria. The next step can be interpreting wireline image logs for each wellbore in the offset wells dataset and producing a mesh of the actual failure that had taken place (post-failure wellbore). This step is illustrated in the wellbore in the right bottom corner in FIG. 37. Finally, all data from the available offset wells can be fed into a machine learning algorithm, which can be decision tree based or a neural network, to enhance the prediction of failure. The final trained model can then be used to generate new predictions of failure for new wells. The main advantage here is that the prediction of failure from the trained model can more accurately describe both the circumferential distribution and radial extension of failure zones as opposed to predicting the critical breakout angle, which is produced from conventional modeling approaches and analytical solutions.

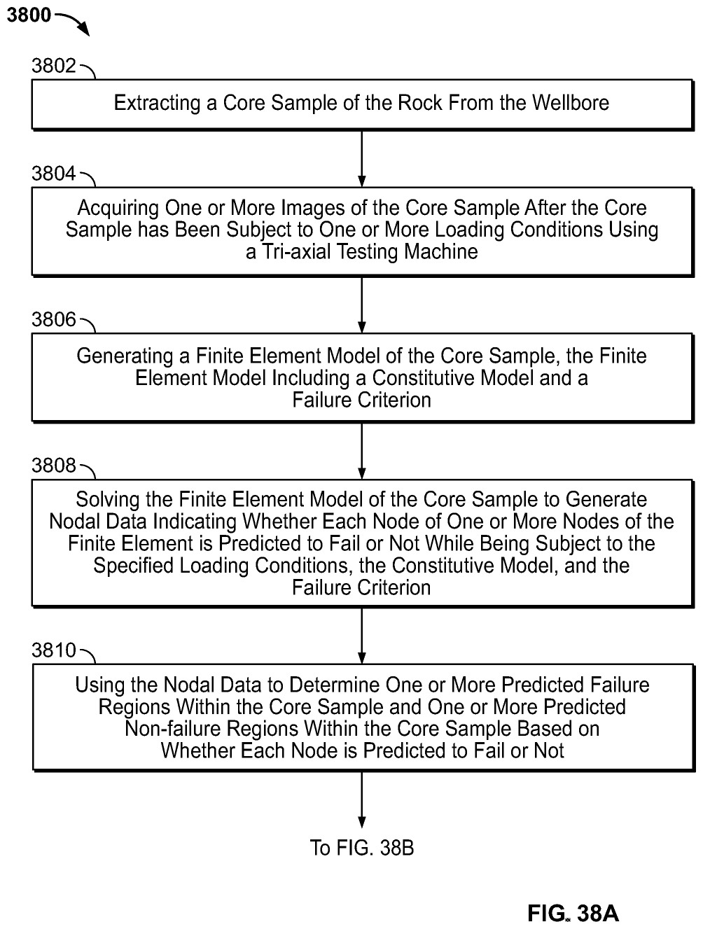

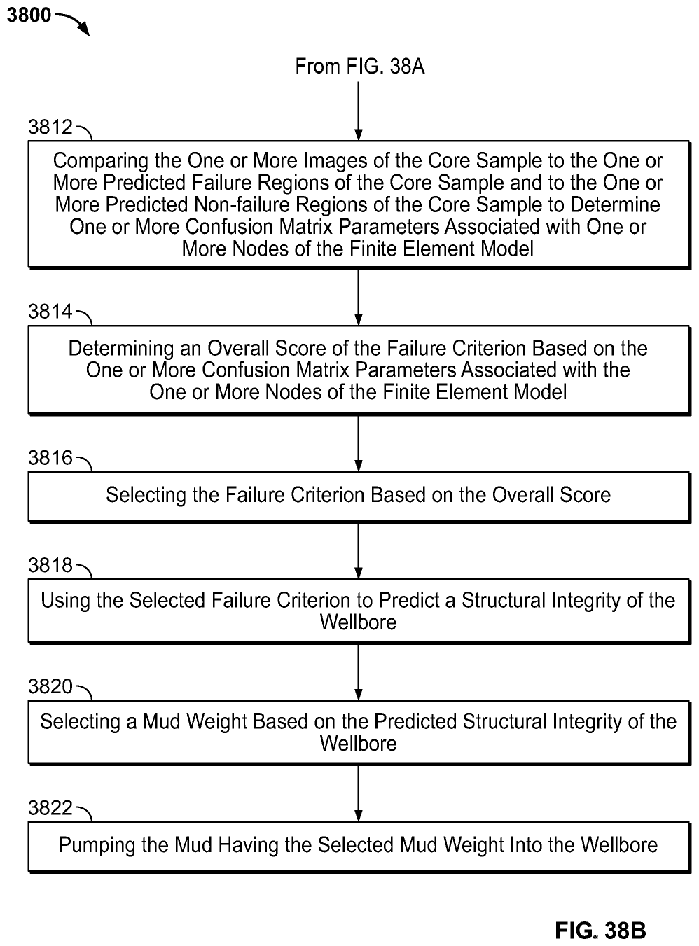

FIGS. 38A and 38B are a flowchart 3800 illustrating an example of a method for determining wellbore integrity based on a statistical analysis of finite element model results combined with rock imaging reference data. The method is used to determine failure criterion for a rock and the failure criterion is used to select and pump a mud into a wellbore to maintain integrity of the wellbore. In some examples, the wellbore is the wellbore 102 described with reference to FIG. 1.

At 3802, a core sample is extracted from the rock of the wellbore. In some examples, a logging tool is lowered into the wellbore and extracts the core sample from the wellbore. In some examples, an engineer extracts the core sample from rock cuttings or chips.

At 3804, one or more images of the core sample are acquired after the core sample has been subject to one or more loading conditions using a tri-axial testing machine. In some examples, the core sample is tested in a tri-axial testing machine as described with reference to FIG. 2 and the computer system images the core sample using a CT-scanner to generate CT scans and/or using a camera to generate DIC images.

In some implementations, a contrast of each of the one or more images is adjusted such that that a wellbore void in each of the one or more images is a first color, and a rock matrix surrounding the wellbore void in each of the one or more images is a second color that is different than the first color. For example, the computer system follows the process described with reference to FIG. 3. In some implementations, after adjusting the contrast of each of the one or more images, a size of each of the one or more images is calibrated based on an actual pixel size measurement from a reference image.

In some implementations, a stress-strain curve of the core sample is acquired while the core sample is subjected to one or more specified loading conditions using the tri-axial testing machine. In some implementations, an unconfined compression strength (UCS) of the core sample is determined based on the acquired stress-strain curve. In some examples, the constitutive model further represents the determined unconfined compression strength.

In some implementations, logging data of the rock is acquired within the wellbore. In some examples, the logging data includes a vertical stress of the rock, a horizontal stress of the rock, image log data of the rock, and a depth within the wellbore associated with the acquisition of the logging data. In some examples, the logging data is used as one or more conditions in a finite element model of the core sample (as described with reference to block 3806). In some implementations, an unconfined compression strength of the rock is predicted based on the acquired logging data as described with reference to FIG. 31. In some examples, the constitutive model further represents the predicted unconfined compression strength.

In some implementations, pixels of each of the one or more images are triangulated. In some examples, the computer system follows the process described with reference to FIGS. 4A-4C to triangulate the pixels of each image. In some examples, each of the one or more images is a cross-section image of the core sample. In some examples, the cross-section is perpendicular to a longitudinal axis of the core sample. In some implementations, a plurality of radii of the core sample are determined based on the triangulated pixels, the plurality of radii of the core sample being a function of circumferential position around the longitudinal axis of the core sample. For example, a plurality of radii of the core sample are determined based on the triangulated pixels according to the process described with reference to FIGS. 4A-4D. In some implementations, triangulating the pixels of each of the one or more images is performed separately along horizontal and vertical directions and then combined to define the triangulated the pixels.

At 3806, a finite element model of the core sample is generated. The finite element model includes a constitutive model and a failure criterion. The constitutive model representing the strain to stress translation necessary for the failure criterion. In some examples, an engineer builds a finite element model the same as or similar to the finite element model 2402 described with reference to FIGS. 24A and 24B on the computer system. In some examples, the computer system builds the finite element model automatically. In some examples, the failure criteria includes a Mohr-Coulomb criterion, a Mogi criterion, a Drucker-Prager criterion, and/or a Lade criterion.

At 3808, the finite element model of the core sample is solved to generate nodal data indicating whether each node of one or more nodes of the finite element model is predicted to fail or not. The finite element model is solved while being subject to specified loading conditions, the constitutive model, and the failure criterion. For example, the computer system follows the process described with reference to FIG. 25 and FIG. 26 to predict failure regions of the core sample by determining which nodes have failed and which nodes have not failed. As used throughout this disclosure, failure/non-failure regions “of” the core sample means both failure/non-failure regions on the boundary of the core sample and failure/non-failure regions within the core sample.

At 3810 the computer system uses (or post-processes) the nodal data of the solved finite element model to determine one or more predicted failure regions of the core sample and one or more predicted non-failure regions of the core sample. In some implementations, determining the one or more predicted failure regions is performed by determining an envelope surrounding each node that is predicted to fail while avoiding each node that is not predicted to fail. In some implementations, determining the one or more predicted non-failure regions is performed by determining an envelope of surrounding each node that is not predicted to fail while avoiding each node that is predicted to fail.

At 3812, the one or more images of the core sample are compared to the one or more predicted failure regions of the core sample and to the one or more predicted non-failure regions of the core sample to determine one or more confusion matrix parameters associated with the one or more nodes of the finite element model. For example, the computer system generates plots similar to or the same as plot 2800 described with reference to FIG. 28 and plot 2700 described with reference to FIG. 27. In some implementations, this involves superimposing the one or more predicted failure regions of the core sample on the one or more images of the core sample.

In some implementations, the confusion matrix parameters represent whether each confusion matrix parameters is true negative, false negative, false positive, or true positive as described with reference to FIG. 28 and Table 1. In some implementations, determining the one or more confusion matrix parameters associated with the one or more nodes of the finite element model includes: (i) perturbing the one or more nodes of the finite element model based on the acquired one or more images and (ii) comparing the perturbed finite element model to the one or more predicted failure regions of the core sample and the one or more predicted non-failure regions of the core sample to determine the one or more confusion matrix parameters associated with the one or more nodes of the finite element model. For example, the computer system performs the process described with reference to FIG. 27.

In some implementations, determining the one or more confusion matrix parameters associated with the one or more nodes of the finite element model includes (i) determining a first set of nodes of the finite element model that are associated with the one or more predicted failure regions of the core sample and associated with one or more failure regions of the one or more images of the core sample and (ii) determining the confusion matrix parameter for the first set of nodes as true positive. For example, the computer system determines nodes within the first set as nodes that fall within the true positive region 2860 as described with reference to FIG. 28 and classifies these nodes as true positive based on Table 1.

In some implementations, determining the one or more confusion matrix parameters associated with the one or more nodes of the finite element model includes (i) determining a second set of nodes of the finite element model that are associated with the one or more predicted non-failure regions of the core sample and associated with one or more non-failure regions of the one or more images of the core sample and (ii) determining the confusion matrix parameter for the second set of nodes as true negative. For example, the computer system determines nodes within the second set as nodes that fall within the true negative region 2880 as described with reference to FIG. 28 and classifies these nodes as true negative based on Table 1.

In some implementations, determining the one or more confusion matrix parameters associated with the one or more nodes of the finite element model includes (i) determining a third set of nodes of the finite element model that are associated with the one or more predicted failure regions of the core sample and associated with the one or more non-failure regions of the one or more images of the core sample and (ii) determining the confusion matrix parameter for the third set of nodes as false positive. For example, the computer system determines nodes within the third set as nodes that fall within the false positive region 2850 as described with reference to FIG. 28 and classifies these nodes as false positive based on Table 1.

In some implementations, determining the one or more confusion matrix parameters associated with the one or more nodes of the finite element model includes (i) determining a fourth set of nodes of the finite element model that are associated with the one or more predicted non-failure regions of the core sample and associated with the one or more failure regions of the one or more images of the core sample and (ii) determining the confusion matrix parameter for the fourth set of nodes as false negative. For example, the computer system determines nodes within the fourth set as nodes that fall within the false negative region 2870 as described with reference to FIG. 28 and classifies these nodes as false negative based on Table 1.

In some implementations, comparing the one or more images of the core sample to the one or more predicted failure regions of the core sample and to the one or more predicted non-failure regions of the core sample includes determining the first set of nodes of the finite element model, the second set of nodes of the finite element model, the third set of nodes of the finite element model, and the fourth set of nodes of the finite element model based on the plurality of radii of the core sample. For example, the statistical algorithm described with reference to FIGS. 30 and 31 use the classification of all nodes to determine the overall F1-score associated with the accuracy of the finite element model and the particular failure criterion used. In some examples, the calibrated size of each of the one or more images is used in the comparison process.

At 3814, an overall score of the failure criterion is determined based on the one or more confusion matrix parameters associated with the one or more nodes of the finite element model. For example, the computer system determines the overall score using the statistical algorithm described with reference to reference to FIGS. 30 and 31.

In some implementations, determining the overall score of the failure criterion based on the one or more confusion matrix parameters associated with the one or more nodes of the finite element model includes (i) determining an overall accuracy of the failure criterion based on the one or more confusion matrix parameters, (ii) determining an overall recall of the failure criterion based on the one or more confusion matrix parameters, and (iii) determining the overall score of the failure criterion based on the precision and the recall. In some implementations, the overall score is independent of a skewness of the one or more confusion matrix parameters toward a particular true negative, false negative, false positive, or true positive result.

At 3816, the failure criterion is selected based on the overall score. In some implementations, selecting the failure criterion based on the overall score includes selecting the failure criterion based on a ranking of the overall scores for the plurality of different failure criterions. For example, the computer system selects the failure criterion by selecting the failure criterion associated with the largest F1-Score as described with reference to FIGS. 30 and 31.

At 3818, the selected failure criterion is used to predict a structural integrity of the wellbore. For example, the computer system and/or an engineer generates a three-dimensional finite element model of the wellbore that includes the selected failure criterion. In some implementations, the three-dimensional finite element model of the wellbore is solved to predict one or more failure regions of the wellbore while being subject to the one or more loading conditions and the selected failure criterion. In some implementations, using the selected failure criterion to predict the structural integrity of the wellbore includes predicting the structural integrity based on the one or more predicted failure regions of the wellbore.

At 3820, a mud weight is selected based on the predicted structural integrity of the wellbore. For example, the computer system selects a mud weight in accordance with the process described with reference to FIGS. 33-35.

At 3822, the mud having the selected mud weight is pumped into the wellbore. For example, a pump located at the surface of the wellbore 102 pumps the selected mud weight into the wellbore 102 to stabilize the wellbore 102 so the well does not become over pressurized and/or under pressurized as described with reference to FIG. 1.

In some implementations, the following steps are repeated by the method: (i) generating the finite element model of the core sample, (ii) solving the finite element model to generate the nodal data, (iii) using the nodal data to determine the one or more predicted failure regions of the core sample and the one or more predicted non-failure regions of the core sample, (iv) comparing the one or more images of the core sample to the one or more predicted failure regions of the core sample to determine one or more confusion matrix parameters associated with the one or more nodes of the finite element model, and (v) determining the overall score of the failure criterion based on the one or more confusion matrix parameters associated with the one or more nodes of the finite element model.

In some implementations, the steps (i)-(v) are repeated for a plurality of different failure criterions to determine an overall score associated with each of the plurality of different failure criterions. In some examples, the plurality of different failure criterions includes at least two of a Mohr-Coulomb failure criterion, a Mogi failure criterion, a Mogi-Coulomb criterion, and a Drucker-Prager failure criterion.

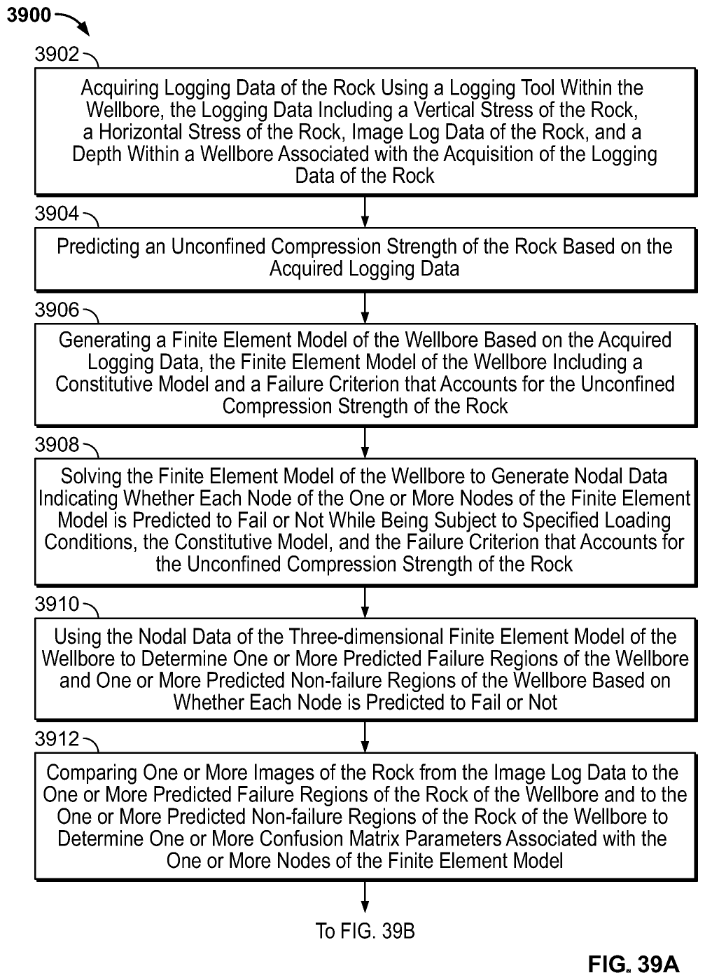

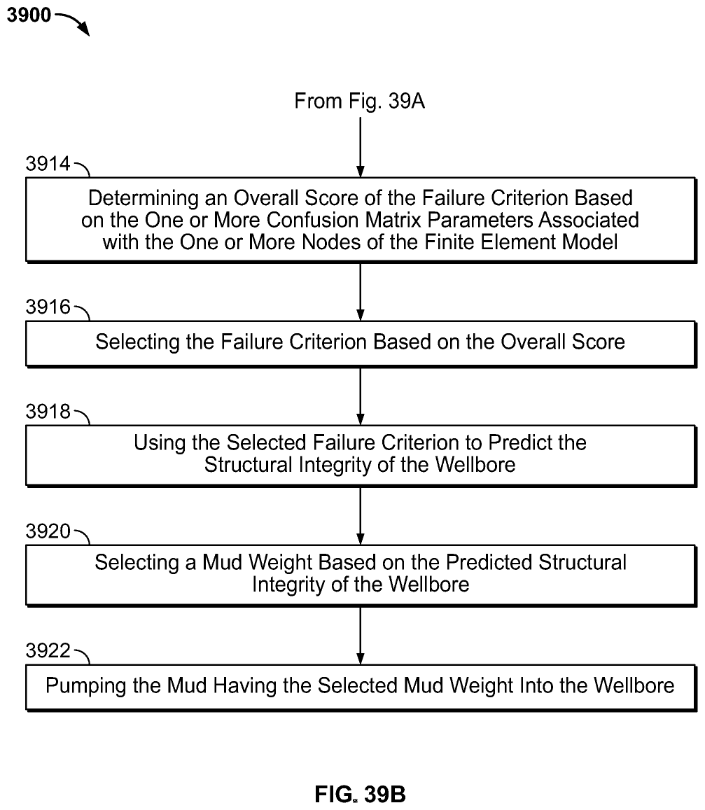

FIGS. 39A and 39B are a flowchart 3900 illustrating an example of a method for determining wellbore integrity based on a statistical analysis of finite element model results combined with rock logging reference data (instead of using lab imaging reference data). The method is used for determining a failure criterion for a rock and the failure criterion is used to select and pump a mud into a wellbore to maintain integrity of the wellbore. In some examples, the computer system 4000 implements one or more steps of the method of flowchart 3900. In some examples, the wellbore is the wellbore 102 described with reference to FIG. 1.

At 3902, logging data of the rock is acquired using a logging tool within the wellbore. The logging data includes a vertical stress of the rock, a horizontal stress of the rock, image log data of the rock, and a depth within a wellbore associated with the acquisition of the logging data of the rock. In some implementations, acquiring the logging data of the rock using the logging tool within the wellbore includes using a micro-imager or micro-scanner tool to acquire resistivity based images of the rock of the wellbore.

At 3904, an unconfined compression strength of the rock is predicted based on the acquired logging data. In some examples, the computer system predicts the unconfined compression strength of the rock as described with reference to FIG. 31.

At 3906, a finite element model of the wellbore is generated based on the acquired logging data. The finite element model of the wellbore includes a constitutive model that represents the unconfined compression strength of the rock and a failure criterion. For example, an engineer builds a finite element model to represent the wellbore. In some examples, this step is the same as or similar to step 3808 described with reference to FIG. 38 except that instead of a core sample being modelled, the wellbore is modelled.

At 3908, the finite element model of the wellbore is solved to predict (i) one or more failure regions of the rock of the wellbore and (ii) one or more non-failure regions of the rock of the wellbore while being subject to one or more loading conditions and the constitutive model that represents the unconfined compression strength of the rock and the failure criterion. In some examples, this step is the same as, or similar to, step 3808 described with reference to FIG. 38.

At 3910 cone or more images of the rock from the image log data are compared to the one or more predicted failure regions of the rock of the wellbore and to the one or more predicted non-failure regions of the rock of the wellbore to determine one or more confusion matrix parameters associated with one or more nodes of the finite element model. In some implementations, determining the one or more confusion matrix parameters associated with one or more nodes of the finite element model includes determining whether nodes associated with the comparison are true positive, true negative, false positive, and false negative in a manner similar to, or the same as, the method described with reference to FIG. 38.

In some implementations, the computer system determines a first set of nodes of the finite element model that are associated with the one or more predicted failure regions of the rock of the wellbore and associated with one or more failure regions of the one or more images of the rock from the image log data and determines the confusion matrix parameter for the first set of nodes as true positive. In some implementations, the computer system determines a second set of nodes of the finite element model that are associated with one or more predicted non-failure regions of the rock of the wellbore and associated with one or more non-failure regions of the one or more images of the rock from the image log data and determines the confusion matrix parameter for the second set of nodes as true negative. In some implementations, the computer system determines a third set of nodes of the finite element model that are associated with the one or more predicted failure regions of the rock of the wellbore and associated with the one or more non-failure regions of the one or more images from the image log data and determines the confusion matrix parameter for the third set of nodes as false positive. In some implementations, the computer system determines a fourth set of nodes of the finite element model that are associated with the one or more predicted non-failure regions of the rock of the wellbore and associated with the one or more failure regions of the one or more images from the image log data and determines the confusion matrix parameter for the fourth set of nodes as false negative.

At 3912, an overall score of the failure criterion is determined based on the one or more confusion matrix parameters associated with the one or more nodes of the finite element model. At 3914, the failure criterion is selected based on the overall score.

At 3916, the selected failure criterion is used to predict a structural integrity of the wellbore. At 3918, a mud weight is selected based on the predicted structural integrity of the wellbore. At 3920 the mud having the selected mud weight is pumped into the wellbore.

FIG. 40 depicts an environment architecture of an example computer-implemented system 4000 (for example, computer system 4000) that can execute implementations of the present disclosure. For example, the computer system 4000 performs the imaging algorithm described with reference to FIGS. 8A-8B, 10A-10B, and 11A-11B, controls a CT-scanner to acquire the CT-scans of the core sample, controls a camera to acquire images of the core sample, controls logging devices to acquire logging data within a wellbore, generates and solves the three-dimensional finite element model described with reference to FIGS. 24A-24B, and performs the statistical analysis to determine the failure criterion as described with FIGS. 38A-38B and FIGS. 39A-39B.

In the depicted example, the example system 4000 includes a client device 4002, a client device 4004, a network 4010 and a cloud environment 4006 and a cloud environment 2908. The cloud environment 4006 may include one or more server devices and databases (for example, processors, memory). In the depicted example, a user 4014 interacts with the client device 4002, and a user 4016 interacts with the client device 4004.

In some examples, the client device 4002 and/or the client device 4004 can communicate with the cloud environment 4006 and/or cloud environment 4008 over the network 4010. The client device 4002 can include any appropriate type of computing device, for example, a desktop computer, a laptop computer, a handheld computer, a tablet computer, a personal digital assistant (PDA), a cellular telephone, a network appliance, a camera, a smart phone, an enhanced general packet radio service (EGPRS) mobile phone, a media player, a navigation device, an email device, a game console, or an appropriate combination of any two or more of these devices or other data processing devices. In some implementations, the network 4010 can include a large computer network, such as a local area network (LAN), a wide area network (WAN), the Internet, a cellular network, a telephone network (e.g., PSTN) or an appropriate combination thereof connecting any number of communication devices, mobile computing devices, fixed computing devices and server systems.

In some implementations, the cloud environment 4006 include at least one server and at least one data store 4020. In the example of FIG. 40 the cloud environment 4006 is intended to represent various forms of servers including, but not limited to, a web server, an application server, a proxy server, a network server, and/or a server pool. In general, server systems accept requests for application services and provides such services to any number of client devices (e.g., the client device 4002 over the network 4010).

The cloud environment 4006 can host applications and databases running on host infrastructure. In some instances, the cloud environment 4006 can include multiple cluster nodes that can represent physical or virtual machines. A hosted application and/or service can run on VMs hosted on cloud infrastructure. In some instances, one application and/or service can run as multiple application instances on multiple corresponding VMs, where each instance is running on a corresponding VM.

What is claimed is:

1. A method for determining a failure criterion and using the failure criterion to predict a structural integrity of a wellbore, the method comprising:
extracting a core sample of a rock from the wellbore;
acquiring one or more images of the core sample after the core sample has been subject to specified loading conditions using a tri-axial testing machine;
generating a finite element model of the core sample, the finite element model including a constitutive model and a failure criterion;
solving the finite element model of the core sample to generate nodal data indicating whether each node of one or more nodes of the finite element model is predicted to fail or not while being subject to the specified loading conditions, the constitutive model, and the failure criterion;
using the nodal data to determine one or more predicted failure regions of the core sample and one or more predicted non-failure regions of the core sample based on whether each node is predicted to fail or not;
comparing the one or more images of the core sample to the one or more predicted failure regions of the core sample and to the one or more predicted non-failure regions of the core sample to determine one or more confusion matrix parameters associated with the one or more nodes of the finite element model;
determining an overall score of the failure criterion based on the one or more confusion matrix parameters associated with the one or more nodes of the finite element model;
selecting the failure criterion based on the overall score; and
using the selected failure criterion to predict the structural integrity of the wellbore.

2. The method of claim 1, further comprising selecting a mud weight based on the predicted structural integrity of the wellbore and pumping a mud having the selected mud weight into the wellbore to provide the predicted stability to the wellbore.

3. The method of claim 1, wherein determining the one or more confusion matrix parameters associated with the one or more nodes of the finite element model comprises determining whether each confusion matrix parameters is true negative, false negative, false positive, or true positive.

4. The method of claim 3, wherein determining the overall score of the failure criterion based on the one or more confusion matrix parameters associated with the one or more nodes of the finite element model comprises:
determining an overall accuracy of the failure criterion based on the one or more confusion matrix parameters;
determining an overall recall of the failure criterion based on the one or more confusion matrix parameters; and
determining the overall score of the failure criterion based on the precision and the overall recall.

5. The method of claim 1, wherein comparing the one or more images of the core sample to the one or more predicted failure regions of the core sample and the one or more predicted non-failure regions of the core sample to determine the one or more confusion matrix parameters associated with the one or more nodes of the finite element model comprises:
perturbing the one or more nodes of the finite element model based on the acquired one or more images; and
comparing the perturbed finite element model to the one or more predicted failure regions of the core sample and the one or more predicted non-failure regions of the core sample to determine the one or more confusion matrix parameters associated with the one or more nodes of the finite element model.

6. The method of claim 1, wherein comparing the one or more images of the core sample to the one or more predicted failure regions of the core sample and the one or more predicted non-failure regions of the core sample to determine the one or more confusion matrix parameters associated with the one or more nodes of the finite element model comprises superimposing the one or more predicted failure regions of the core sample on the one or more images of the core sample.

7. The method of claim 1, wherein comparing the one or more images of the core sample to the one or more predicted failure regions of the core sample and the one or more predicted non-failure regions of the core sample to determine the one or more confusion matrix parameters associated with the one or more nodes of the finite element model comprises:
determining a first set of nodes of the finite element model that are associated with the one or more predicted failure regions of the core sample and associated with one or more failure regions of the one or more images of the core sample;
determining the confusion matrix parameter for the first set of nodes as true positive;
determining a second set of nodes of the finite element model that are associated with the one or more predicted non-failure regions of the core sample and associated with one or more non-failure regions of the one or more images of the core sample; and
determining the confusion matrix parameter for the second set of nodes as true negative.

8. The method of claim 7, wherein comparing the one or more images of the core sample to the one or more predicted failure regions of the core sample and the one or more predicted non-failure regions of the core sample to determine the one or more confusion matrix parameters associated with the one or more nodes of the finite element model comprises:
determining a third set of nodes of the finite element model that are associated with the one or more predicted failure regions of the core sample and associated with the one or more non-failure regions of the one or more images of the core sample;
determining the confusion matrix parameter for the third set of nodes as false positive;
determining a fourth set of nodes of the finite element model that are associated with the one or more predicted non-failure regions of the core sample and associated with the one or more failure regions of the one or more images of the core sample; and
determining the confusion matrix parameter for the fourth set of nodes as false negative.

9. The method of claim 8, further comprising:
triangulating pixels of each of the one or more images, wherein each of the one or more images is a cross-section image of the core sample, the cross-section being perpendicular to a longitudinal axis of the core sample;
determining a plurality of radii of the core sample based on the triangulated pixels, the plurality of radii of the core sample being a function of circumferential position around the longitudinal axis of the core sample;
wherein comparing the one or more images of the core sample to the one or more predicted failure regions of the core sample and to the one or more predicted non-failure regions of the core sample comprises determining the first set of nodes of the finite element model, the second set of nodes of the finite element model, the third set of nodes of the finite element model, and the fourth set of nodes of the finite element model based on the plurality of radii of the core sample.

10. The method of claim 9, further comprising:
adjusting a contrast of each of the one or more images such that that a wellbore void in each of the one or more images is a first color, and a rock matrix surrounding the wellbore void in each of the one or more images is a second color that is different than the first color; and
after adjusting the contrast of each of the one or more images, calibrating a size of each of the one or more images based on an actual pixel size measurement from a reference image,
wherein comparing the one or more images of the core sample to the one or more predicted failure regions of the core sample and to the one or more predicted non-failure regions of the core sample comprises comparing the one or more images of the core sample to the one or more predicted failure regions of the core sample and to the one or more predicted non-failure regions of the core sample based on the calibrated size of each of the one or more images.

11. The method of claim 9, wherein triangulating the pixels of each of the one or more images is performed separately along horizontal and vertical directions and then combined to define the triangulated the pixels.

12. The method of claim 1, further comprising:
repeating the steps of: (i) generating the finite element model of the core sample, (ii) solving the finite element model to generate the nodal data, (iii) using the nodal data to determine the one or more predicted failure regions of the core sample and the one or more predicted non-failure regions of the core sample, (iv) comparing the one or more images of the core sample to the one or more predicted failure regions of the core sample to determine one or more confusion matrix parameters associated with the one or more nodes of the finite element model, and (v) determining the overall score of the failure criterion based on the one or more confusion matrix parameters associated with the one or more nodes of the finite element model,
wherein steps (i)-(v) are repeated for a plurality of different failure criterions to determine an overall score associated with each of the plurality of different failure criterions, the plurality of different failure criterions including at least two of a Mohr-Coulomb failure criterion, a Mogi failure criterion, a Mogi-Coulomb criterion, and a Drucker-Prager failure criterion,
wherein selecting the failure criterion based on the overall score comprises selecting the failure criterion based on a ranking of the overall scores for the plurality of different failure criterions.

13. The method of claim 1, wherein using the selected failure criterion to predict the structural integrity of the wellbore comprises:
generating a three-dimensional finite element model of the wellbore, the three-dimensional finite element model of the wellbore including the constitutive model and the selected failure criterion;
solving the three-dimensional finite element model of the wellbore to generate nodal data indicating whether each node of one or more nodes of the finite element is predicted to fail or not while being subject to the selected failure criterion;
using the nodal data of the three-dimensional finite element model of the wellbore to determine one or more predicted failure regions of the wellbore and one or more predicted non-failure regions of the wellbore based on whether each node is predicted to fail or not; and
predicting the structural integrity of the wellbore based on the one or more predicted failure regions of the wellbore.

14. The method of claim 1, further comprising:
acquiring a stress-strain curve of the core sample while the core sample is subjected to one or more loading conditions using the tri-axial testing machine; and
determining an unconfined compression strength of the core sample based on the acquired stress-strain curve, the constitutive model further representing the determined unconfined compression strength.

15. The method of claim 1, further comprising:
acquiring logging data of the rock, the logging data including a vertical stress of the rock, a horizontal stress of the rock, image log data of the rock, and a depth within the wellbore associated with the acquisition of the logging data; and
predicting an unconfined compression strength of the rock based on the acquired logging data, the constitutive model further representing the determined unconfined compression strength.

16. The method of claim 1, wherein acquiring the one or more images of the core sample after the core sample has been subject to specified loading conditions using the tri-axial testing machine comprises performing a CT-scan on the core sample to generate the one or more images of the core sample.

17. A method for determining a failure criterion and using the failure criterion to determine a structural integrity of a wellbore, the method comprising:
acquiring logging data of a rock surrounding the wellbore using a logging tool within the wellbore, the logging data including a vertical stress of the rock, a horizontal stress of the rock, image log data of the rock, and a depth within a wellbore associated with the acquisition of the logging data of the rock;
predicting an unconfined compression strength of the rock based on the acquired logging data;
generating a finite element model of the wellbore based on the acquired logging data, the finite element model of the wellbore including a constitutive model and a failure criterion that accounts for the unconfined compression strength of the rock;
solving the finite element model of the wellbore to generate nodal data indicating whether each node of one or more nodes of the finite element model is predicted to fail or not while being subject to specified loading conditions, the constitutive model and the failure criterion that accounts for the unconfined compression strength of the rock;
using the nodal data to determine one or more predicted failure regions of the wellbore and one or more non-failure regions of the wellbore based on whether each node is predicted to fail or not;
comparing one or more images of the rock from the image log data to the one or more predicted failure regions of the rock of the wellbore and to the one or more predicted non-failure regions of the rock of the wellbore to determine one or more confusion matrix parameters associated with the one or more nodes of the finite element model;
determining an overall score of the failure criterion based on the one or more confusion matrix parameters associated with the one or more nodes of the finite element model;
selecting the failure criterion based on the overall score;
using the selected failure criterion to predict the structural integrity of the wellbore;
selecting a mud weight based on the predicted structural integrity of the wellbore; and
pumping the mud having the selected mud weight into the wellbore.

18. The method of claim 17, wherein comparing the one or more images of the rock from the image log data to the one or more predicted failure regions of the rock of the wellbore and to the one or more predicted non-failure regions of the rock of the wellbore to determine the one or more confusion matrix parameters associated with one or more nodes of the finite element model comprises:
determining a first set of nodes of the finite element model that are associated with the one or more predicted failure regions of the rock of the wellbore and associated with one or more failure regions of the one or more images of the rock from the image log data;
determining the confusion matrix parameter for the first set of nodes as true positive;
determining a second set of nodes of the finite element model that are associated with one or more predicted non-failure regions of the rock of the wellbore and associated with one or more non-failure regions of the one or more images of the rock from the image log data; and
determining the confusion matrix parameter for the second set of nodes as true negative.

19. The method of claim 18, wherein comparing the one or more images of the rock from the image log data to the one or more predicted failure regions of the rock of the wellbore and to the one or more predicted non-failure regions of the rock of the wellbore to determine the one or more confusion matrix parameters associated with one or more nodes of the finite element model comprises:
determining a third set of nodes of the finite element model that are associated with the one or more predicted failure regions of the rock of the wellbore and associated with the one or more non-failure regions of the one or more images from the image log data;
determining the confusion matrix parameter for the third set of nodes as false positive;
determining a fourth set of nodes of the finite element model that are associated with the one or more predicted non-failure regions of the rock of the wellbore and associated with the one or more failure regions of the one or more images from the image log data; and
determining the confusion matrix parameter for the fourth set of nodes as false negative.

20. The method of claim 17, wherein acquiring the logging data of the rock using the logging tool within the wellbore comprises using a micro-imager or micro-scanner tool to acquire resistivity based images of the rock of the wellbore, and the failure criterion includes at least one of a Mohr-Coulomb failure criterion, a Mogi failure criterion, a Mogi-Coulomb criterion, and a Drucker-Prager failure criterion.
